(19) United States
(12) Patent Application Publication
(10) Pub. No.: US 2006/0167025 A1
(43) Pub. Date: Jul. 27, 2006

(54) Tricyclic amino alcohols, processes for synthesis of same and use of same as anti-inflammatory drugs

(76) Inventors: Markus Berger, Berlin (DE); Norbert Schmees, Berlin (DE); Heike Schaecke, Berlin (DE); Stefan Baurle, Berlin (DE); Hartmut Rehwinkel, Berlin (DE); Anne Mengel, Berlin (DE); Konrad Krolikiewicz, Berlin (DE); Danja Grossbach, Berlin (DE); David Voigtlaender, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/312,655

(22) Filed: Dec. 21, 2005

Related U.S. Application Data
(60) Provisional application No. 60/642,519, filed on Jan. 11, 2005.

(30) Foreign Application Priority Data
Dec. 22, 2004 (DE) 102004063227.8

(51) Int. Cl. A61K 31/517; A61K 31/403; A61K 31/381; A61K 31/343

(52) U.S. Cl. 514/266.2; 514/411; 514/443; 514/468; 544/284; 548/440; 549/49; 549/433

(57) ABSTRACT

The invention relates to tricyclic amino alcohols of general formula (I) 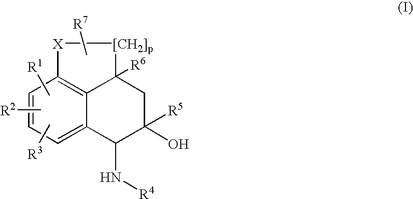 method for synthesis of same and use of same as anti-inflammatory agents.

This application claims the benefit of the filing date of U.S. Provisional Application Ser. No. 60/642,519 filed Jan. 11, 2005 which is incorporated by reference herein.

The present invention relates to tricyclic amino alcohols, processes for synthesis of same, and use of same as anti-inflammatory drugs.

Open-chain non-steroidal anti-inflammatory drugs are known from the related art documents WO 02/10143 and WO 03/082827. In experiments, these compounds have shown a dissociation between anti-inflammatory effects and adverse metabolic effects and have been found to be superior or at least equivalent to the non-steroidal glucocorticoids described previously.

However, these state-of-the-art-compounds still have some disadvantages, so therefore, those skilled in the art are motivated to continue searching for novel compounds that will bind to the glucocorticoid receptor.

Compounds having effects comparable to those of the compounds described in the state of the art have now been discovered.

The present invention relates to compounds of the general formula (I) 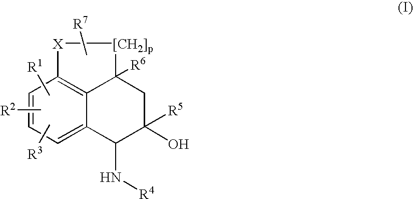
where
R¹ and R² independently of one another denote a hydrogen atom, a hydroxyl group, a halogen atom, an optionally substituted (C₁-C₁₀) alkyl group, a (C₁-C₁₀) alkoxy group, a (C₁-C₁₀) alkylthio group, a (C₁-C₅) perfluoroalkyl group, a cyano group, a nitro group or R¹ and R² together may form a group selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, —NH—N═CH—, where n=1 or 2 and the terminal atoms are linked to directly vicinal ring carbons or NR⁸R⁹, where R⁸ and R⁹ independently of one another may denote hydrogen, C₁-C₅ alkyl or (CO)—C₁-C₅ alkyl,
R³ denotes a hydrogen atom, a hydroxyl group, a halogen atom, an optionally substituted (C₁-C₁₀) alkyl group, an (C₁₁-C₁₀) alkoxy group, a (C₁-C₁₀) alkylthio group, a (C₁-C₅) perfluoroalkyl group, a cyano group,
R⁴ denotes a C₁-C₁₀ alkyl group or a C₁-C₁₀ alkyl group substituted by one or more groups selected from 1 to 3 hydroxyl groups, 1 to 3 (C₁-C₅) alkoxy groups; an optionally substituted (C₃-C₇) cycloalkyl group, an optionally substituted heterocyclyl group, an optionally substituted aryl group; a mono- or bicyclic heteroaryl group optionally containing 1 to 4 nitrogen atoms and/or 1 to 2 oxygen atoms and/or 1 to 2 sulfur atoms and/or 1 to 2 keto groups, optionally substituted by one or more groups selected from (C₁-C₅) alkyl groups (which may optionally be substituted by 1 to 3 hydroxyl or 1 to 3 COOR¹⁰ groups where R¹⁰ denotes C₁-C₆ alkyl or benzyl), (C₁-C₅) alkoxy groups, hydroxyl groups, halogen atoms, (C₁-C₃) exoalkylidene groups, where this group may be linked to the amine of the ring system at any position and may optionally be hydrogenated at one or more positions,
R⁵ denotes a (C₁-C₅) alkyl group or an optionally partially or completely fluorinated (C₁-C₅) alkyl group, a (C₃-C₇) cycloalkyl group, a (C₃-C₇) cycloalkyl(C₁-C₈)alkyl group, a (C₃-C₇) cycloalkyl(C₂-C₈)alkenyl group, a heterocyclyl group, a heterocyclyl(C₁-C₈)alkyl group, heterocyclyl(C₂-C₈)alkenyl group, an aryl group, an aryl(C₁-C₈)alkyl group, (an aryl(C₂-C₈)alkenyl group, an aryl(C₂-C₈)alkynyl group; a mono- or bicyclic heteroaryl group containing one or more nitrogen atoms and/or oxygen atoms and/or sulfur atoms and optionally substituted by one or more keto groups, (C₁-C₅) alkyl groups, (C₁-C₅) alkoxy groups, halogen atoms, (C₁-C₃) exoalkylidene groups;
a heteroaryl(C₁-C₈)alkyl group or a heteroaryl(C₂-C₈)alkenyl group, where these groups may be linked to the chromene system at any position
and may optionally be hydrogenated in one or more positions,
R⁶ and R⁷ independently of one another denote a hydrogen atom, a halogen atom, a (C₁-C₅) alkyl group, which may be substituted with OR⁸, SR⁸, NR⁸R⁹,
p is 1 to 3 and
X is an oxygen atom, a sulfur atom, a CH₂ group or an NR⁹ group.

The present invention also relates to stereoisomers of general formula (I) where R¹ and R² independently of one another denote a hydrogen atom, a hydroxyl group, a halogen atom, an optionally substituted (C₁-C₁₀) alkyl group, a (C₁-C₁₀) alkoxy group, a (C₁-C₁₀) alkylthio group, a (C₁-C₅) perfluoroalkyl group, a cyano group, a nitro group or R¹ and R² together may form a group selected from the groups —O—(CH₂)ₙ—O—, —O—(CH₂)ₙ—CH₂—, —O—CH═CH—, —(CH₂)ₙ₊₂—, where n=1 or 2 and the terminal atoms are linked to directly vicinal ring carbons, or NR⁸R⁹, where R⁸ and R⁹ independently of one another denote hydrogen, C₁-C₅ alkyl or (CO)—C₁-C₅ alkyl,

R³ denotes a hydrogen atom, a hydroxyl group, a halogen atom, an optionally substituted (C₁-C₁₀) alkyl group, an (C₁-C₁₀) alkoxy group, a (C₁-C₁₀) alkylthio group, a (C₁-C₅) perfluoroalkyl group, a cyano group,

R⁴ denotes a C₁-C₁₀ alkyl group, a substituted C₁-C₁₀ alkyl group with one or more groups as substituents selected from 1 to 3 hydroxyl groups, halogen atoms, 1 to 3 (C₁-C₅) alkoxy groups; an optionally substituted phenyl group; a mono- or bicyclic heteroaryl group containing 1 to 3 nitrogen atoms and/or 1 to 2 oxygen atoms and/or 1 to 2 sulfur atoms and/or 1 to 2 keto groups, optionally substituted by 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups, where these groups may be linked to the amine of the ring system at any position and may optionally be hydrogenated at one or more positions,

R⁵ denotes a (C₁-C₅) alkyl group or an optionally partially or completely fluorinated (C₁-C₅) alkyl group, an aryl group, an aryl(C₁-C₈)alkyl group, an aryl(C₂-C₈)alkenyl group, a (C₃-C₇)cycloalkyl group, a (C₃-C₇)cycloalkyl(C₁-C₈)alkyl group, a (C₃-C₇)cycloalkyl(C₂-C₈) alkenyl group

R⁶ and R⁷ independently of one another denote a hydrogen atom, a halogen atom, a methyl or ethyl group, which may be substituted with OR⁸, SR⁸, N(R⁹)₂,

p is 1 to 3 and

X is an oxygen atom, a sulfur atom, a CH₂ or an NR⁹ group.

The present invention also relates to stereoisomers of general formula (I) wherein R¹ and R² independently of one another denote a hydrogen atom, a hydroxyl group, a halogen atom, an optionally substituted (C₁-C₅) alkyl group, a (C₁-C₅) alkoxy group, or R¹ and R² together may denote a group selected from the groups —O—(CH₂)ₙ—O—, —O—(CH₂)ₙ—CH₂—, —O—CH═CH—, —(CH₂)ₙ₊₂—,

where n=1 or 2 and the terminal atoms are linked to directly vicinal ring carbons,

R³ denotes a hydrogen atom, a hydroxyl group, a halogen atom, an optionally substituted (C₁-C₁₀) alkyl group or a (C₁-C₁₀) alkoxy group,

R⁴ denotes a (C₁-C₁₀) alkyl group or a substituted (C₁-C₁₀) alkyl group with as substituents 1 to 3 hydroxyl groups or halogen atoms; a phenyl, naphthyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group, optionally substituted by one or more groups selected from 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups,

where these groups may be linked to the amine of the

ring system at any position and may optionally be hydrogenated at one or more positions,

R⁵ denotes a (C₁-C₅) alkyl group or an optionally partially or completely fluorinated (C₁-C₅) alkyl group,

R⁶ and R⁷ independently of one another denote a hydrogen atom, a halogen atom, a methyl or ethyl group, which may be substituted with OR⁸, SR⁸, N(R⁹)₂, where R⁸ and R⁹ independently of one another may denote hydrogen, C₁-C₅ alkyl or (CO)—C₁-C₅ alkyl,

p is 1, 2 or 3 and

X is an oxygen atom, a sulfur atom, a CH₂ or an NR⁹ group.

A preferred embodiment of the present invention relates to stereoisomers of general formula (I) according to Claim 1,

wherein

R¹, R² and R³ independently of one another denote a hydrogen atom, a hydroxyl group, a halogen atom, an optionally substituted (C₁-C₁₀) alkyl group, a (C₁-C₁₀) alkoxy group, a cyano group,

R⁴ denotes a mono- or bicyclic heteroaryl group containing 1 to 3 nitrogen atoms and/or 1 to 2 oxygen atoms and/or 1 to 2 sulfur atoms and/or 1 to 2 keto groups and optionally substituted by one or more groups selected from 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups,

where this group may be linked to the amine of the ring system at any position and may optionally be hydrogenated at one or more positions,

R⁵ denotes a (C₁-C₅) alkyl group or an optionally partially or completely fluorinated (C₁-C₅) alkyl group,

R⁷ and R⁸ [sic; R⁶ and R⁷]— independently of one another denote a hydrogen atom, a halogen atom, a methyl or ethyl group,

p is 1 or 2

X is an oxygen or sulfur atom.

Another embodiment of the present invention relates to stereoisomers of general formula (I) wherein

R¹, R² and R³ independently of one another denote a hydrogen atom, a hydroxyl group, a halogen atom, an optionally substituted (C₁-C₁₀) alkyl group, a (C₁-C₁₀) alkoxy group, a cyano group,

R⁴ denotes a phenyl, naphthyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group, optionally substituted by one or more groups selected from 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups, where these groups may be linked to the amine of the ring system at any position and may optionally be hydrogenated at one or more positions,

R⁵ denotes a (C₁-C₅) alkyl group or an optionally partially or completely fluorinated (C₁-C₅) alkyl group,

R⁶ and R⁷ independently of one another denote a hydrogen atom, a halogen atom, a methyl or ethyl group,

p is 1 or 2 and

X is an oxygen atom or a sulfur atom.

An especially preferred embodiment of the present invention relates to stereoisomers according to the Claims 1 to 5, wherein

R¹, R² and R³ independently of one another denote a hydrogen atom or a halogen atom,

R⁴ denotes a bicyclic heteroaryl group optionally containing 1 to 2 nitrogen atoms and/or 1 keto group and optionally substituted by one or more groups selected from (C₁-C₅) alkyl groups, hydroxyl groups, halogen atoms, where this group may be linked to the amine of the ring system at any position and may optionally be hydrogenated at one or more positions,

R⁵ denotes a fluorinated (C₁-C₃) alkyl group,

R⁶ and R⁷ independently of one another denote a hydrogen atom or a (C₁-C₂) alkyl group,

p is 2 and

X is an oxygen or sulfur atom.

An especially preferred embodiment of the present invention relates to stereoisomers of general formula (I) wherein

R¹, R² and R³ independently of one another denote a hydrogen atom or a halogen atom,

R⁴ denotes a phthalazinonyl, quinolinyl, isoquinolinyl, isoquinolonyl, quinolonyl, quinazolinyl, dihydroindolonyl, indazolyl group optionally substituted by one or more groups selected from 1 to 2 keto groups, 1 to 2 (C₁-C₅) alkyl groups, 1 to 3 hydroxyl groups or 1 to 3 halogen atoms,

where these groups may be linked to the amine of the ring system at any position and may optionally be hydrogenated at one or more positions,

R⁵ is a fluorinated (C₁-C₃) alkyl group,

R⁶ and R⁷ independently of one another denote a hydrogen atom or a (C₁-C₂) alkyl group,

p is 2 and

X is an oxygen or sulfur atom.

An especially preferred embodiment of the present invention relates to stereoisomers of general formula (I), wherein

R¹, R² and R³ independently of one another denote a hydrogen atom or a halogen atom,

R⁴ denotes a phthalazinonyl, quinolinyl, quinolonyl, quinazolinyl group optionally substituted by one or more groups selected from 1 to 2 keto groups, 1 to 2 (C₁-C₅) alkyl groups, 1 to 3 hydroxyl groups or 1 to 3 halogen atoms, where these groups may be linked to the amine of the ring system at any position and

may optionally be hydrogenated at one or more positions,

R⁵ is a fluorinated (C₁-C₃) alkyl group,

R⁶ and R⁷ independently of one another denote a hydrogen atom or a (C₁-C₂) alkyl group,

p is 2 and

X is an oxygen or sulfur atom.

The third condensed ring containing X, [(CH₂)p] and the substituent R⁷ may be a 5-, 6- or 7-membered ring, so p may assume values of 1 to 3; p=2 is preferred.

X may denote an oxygen atom, a sulfur atom, an NR⁹ group or a CH₂ group. The oxygen atom and the sulfur atom are preferred; the oxygen atom is especially preferred.

The term halogen atom or halogen refers to a fluorine, chlorine, bromine or iodine atom A fluorine, chlorine or bromine atom is preferred.

The alkyl groups R¹, R², R³, R⁵, R⁸ and R⁹ may be linear or branched and may stand for a methyl, ethyl, n-propyl, isopropyl, n-butyl, isobutyl, tert-butyl or n-pentyl, 2,2-dimethylpropyl, 2-methylbutyl or 3-methylbutyl group, for example. A C₁ to C₃ alkyl group is preferred. These groups may optionally be substituted by a group selected from 1 to 3 hydroxyl groups, 1 to 3 halogen atoms, 1 to 3 (C₁-C₃) alkoxy and/or 1 to 3 COOR¹⁰ groups. Hydroxyl groups are preferred.

The alkyl group R⁴ has the meanings given in the preceding paragraph, but the possible substituents may be selected from the group consisting of hydroxy, halogen, (C₁-C₅) alkoxy.

The alkyl groups R⁶ and R⁷ have the meanings given in the preceding paragraph but the possible substituents are selected from the groups OR⁹, SR⁹ and NR⁸R⁹, where R⁸ and R⁹ denote hydrogen, C₁-C₅ alkyl or (CO)C₁-C₅ alkyl and alkyl is again defined as above. Especially preferred for R⁶ and R⁷ are a hydrogen atom and the unsubstituted C₁-C₃ alkyl group, a hydrogen atom and a methyl group being especially preferred.

The alkoxy groups may be linear or branched and may denote, for example, a methoxy, ethoxy, n-propoxy, isopropoxy, n-butoxy, isobutoxy, tert-butoxy or n-pentoxy, 2,2-dimethylpropoxy, 2-methylbutoxy or 3-methylbutoxy group. A methoxy group or an ethoxy group is preferred.

The alkylthio groups may be linear or branched and include a methylthio, ethylthio, n-propylthio, isopropylthio, n-butylthio, isobutylthio, tert-butylthio or n-pentylthio, 2,2-dimethylpropylthio, 2-methylbutylthio or 3-methylbutylthio group. A methylthio or ethylthio group is preferred.

Examples of a partially or completely fluorinated alkyl group, which may be linear or branched, include the following partially or completely fluorinated groups: fluoromethyl, difluoromethyl, trifluoromethyl, fluoroethyl, 1,1-difluoroethyl, 1,2-difluoroethyl, 1,1,1-trifluoroethyl, tetrafluoroethyl, pentafluoroethyl, C₃F₇, C₃H₂F₅, C₄F₉, C₅F₁₁. Of these, the trifluoromethyl and pentafluoroethyl groups are preferred, the trifluoromethyl group being especially preferred. The reagents are commercially available or the published syntheses of the respective reagents belong to the state of the art,

The aryl substituents R¹ and R² may form a ring, by the two aryl substituents forming a chain selected from the groups O—(CH₂)ₙ-0, O—(CH₂)ₙ—CH₂, O—CH═CH, (CH₂)ₙ₊₂, NH—(CH₂)ₙ₊₁, N(C₁-C₃-alkyl)-(CH₂)ₙ₊₁, NH—N═CH, where n=1 or 2. The terminal atoms of the groups listed above are linked to directly vicinal aryl ring carbons, forming an annellated ring.

The substituent NR⁸R⁹ denotes, for example NH₂, NH(CH₃), N(CH₃)₂, NH(C₂H₅), N(C₂H₅)₂, NH(C₃H₇), N(C₃H₇)₂, NH(C₄H₉), N(C₄H₉)₂, NH(C₅H₁₁), N(C₅H₁₁)₂, NH(CO)CH₃, NH(CO)C₂H₅, NH(CO)C₃H₇, NH(CO)C₄H₉, NH(CO)C₅H₁₁.

The cycloalkyl group denotes a saturated cyclic group with 3 to 7 ring carbons, optionally substituted with one or more groups selected from hydroxyl group, halogen atoms, (C₁-C₅) alkyl groups, (C₁-C₅) alkoxy groups, e.g., cyclopropyl, methylcyclopropyl, cyclobutyl, methylcyclobutyl, cyclopentyl, methylcyclopentyl, cyclohexyl, methylcyclohexyl, cycloheptyl, methylcycloheptyl.

The cycloalkylalkyl group denotes, for example, (CH₂) cycloalkyl, (C₂H₄) cycloalkyl, (C₃H₆) cycloalkyl, (C₄H₈) cycloalkyl, (C₅H₁₀) cycloalkyl, where cycloalkyl is defined as described above.

Cycloalkylalkenyl group denotes, for example, (CH═CH)-cycloalkyl, [C(CH₃)═CH]-cycloalkyl, [CH═C(CH₃)]-cycloalkyl, (CH═CH—CH₂)-cycloalkyl, (CH₂—CH═CH)-cycloalkyl, (CH═CH—CH₂—CH₂)-cycloalkyl, (CH₂—CH═CH—CH₂)-cycloalkyl, (CH₂—CH₂—CH═CH)-cycloalkyl, (C(CH₃)═CH—CH₂)-cycloalkyl, (CH═C(CH₃)—CH₂)-cycloalkyl.

A (C₁-C₃)-exoalkylidene group is to be understood as being a group linked to the system (ring or chain) by an exo double bond. Exomethylene is preferred.

The heterocyclyl group is not aromatic and may be, for example, pyrrolidine, imidazolidine, pyrazolidine, piperidine. Suitable substituents include hydroxyl groups, halogen atoms, (C₁-C₅) alkyl group and (C₁-C₅) alkoxy groups.

Heterocyclylalkyl groups are to be understood as heterocyclyl group attached to the basic structure by a C₁-C₅ alkyl group, where the alkyl group may be linear or branched.

Heterocyclylalkenyl groups are heterocyclyl groups attached to the structure by an unsaturated C₂-C₅ alkyl group, where the alkenyl groups may be linear or branched.

The aryl group R⁴ and R⁵ may denote phenyl or naphthyl. Suitable substituents for both groups include C₁-C₃ alkyl, hydroxy, C₁-C₃ alkoxy, C₁-C₃ alkylthio, halogen, cyano, COO(C₁-C₅) alkyl, COOH, NR⁹R¹⁰ and nitro. The degree of substitution may be one or more, and there may be several identical or different substituents. Monosubstituted or disubstituted phenyl and naphthyl groups R⁴ are preferred.

The aryl groups may be partially hydrogenated and then in addition to or as an alternative to the substituents mentioned above, they may also have keto, (C₁-C₃)-exoalkylidene. The term partially hydrogenated phenyl is to be understood to refer to cyclohexadienyl, cyclohexenyl, cyclohexyl. For example, a partially hydrogenated substituted naphthalene system would include 1-tetralone or 2-tetralone.

The arylalkyl group is an aryl group attached to a basic structure by a C₁-C₈ alkyl group, where the alkyl group may be linear or branched. Examples include benzyl and phenethylene.

An arylalkenyl group is an aryl group attached to a basic structure by a C₂-C₈ alkenyl group, where the alkenyl group may be linear or branched.

The arylalkynyl group is an aryl group attached to the basic structure by a C₂-C₈ alkynyl group, where the alkynyl group may be linear or branched.

Mono- or bicyclic heteroaryl groups R⁴ and R⁵ that may be hydrogenated at one or more positions include all mono- and bicyclic aromatic ring systems having at least one heteroatom and at most 7 heteroatoms. Preferred examples include ring systems with 1 to 5 heteroatoms. Heteroatoms include 1 to 4 nitrogen atoms, 1 to 2 oxygen atoms and 1 to 2 sulfur atoms, which may occur in all subcombinations in the ring system as long as they do not exceed the number specified for the particular heteroatom and the sum does not exceed the maximum number of 7 heteroatoms. Examples include compounds of formula (I) in which R⁴ or R⁵ denotes furanyl, thienyl, pyrazolyl, pyrrolyl, oxazolyl, thiazolyl, imidazolyl, isoxazolyl, isothiazolyl, oxadiazolyl, triazolyl, tetrazolyl, thiadiazolyl, pyridyl, pyrimidinyl, pyridazinyl, pyrazinyl, triazinyl, azaindolizinyl, phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzimidazolyl, indolizinyl, isobenzofuranyl, azaindolyl, azaisoindolyl, furanopyridyl, furanopyrimidinyl, furanopyrazinyl, furanopyidazinyl, dihydrobenzofuranyl, dihydrofuranopyridyl, dihydrofuranopyrimidinyl, dihydrofuranopyrazinyl, dihydrofuranopyridazinyl, dihydrobenzofuranyl, dihydroisoquinolinyl, dihydroquinolinyl benzoxazinonyl; phthalazinonyl, quinolinyl, isoquinolinyl, quinolonyl, isoquinolonyl, quinazolinyl, quinoxalinyl, cinnolinyl, phthalazinyl, 1,7- or 1,8-naphthyridinyl, which belong to the present invention and constitute a special embodiment of the present invention. If the heteroaryl groups contain a keto group, they may assume different positions within the ring system. All chemically possible regioisomers are included in this definition, e.g., “quinolonyl” includes both quinolin-2(1H)-one and quinolin-3(4H)-one and quinolin-4(1H)-one.

If the heteroaryl groups are partially or completely hydrogenated, then compounds of formula (I) belonging to the present invention include those in which R⁴ denotes tetrahydropyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1-H-pyridin-4-ylidenaminyl, chromanyl, thiochromanyl, decahydroquinolinyl, tetrahydroquinolinyl, dihydroquinolinyl, 5,6,7,8-tetrahydro-1H-quinolin-4-onyl, decahydroisoquinolinyl, tetrahydroisoquinolinyl, dihydroisoquinolinyl, 3,4-dihydro-2Hbenz[1,4]oxazinyl, 1,2-dihydro[1,3]benzoxazin-4-onyl, 3,4-dihydrobenz[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-1H-quinoxalin-2-onyl, 2,3-1,2,3,4-tetrahydro[1,5]naphthyridinyl, dihydro-1H-[1,5]naphthyridyl, 1H-[1,5]naphthyrid-4-onyl, 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, 2,2-dihydro-1H-pyrrolo[2,3-b]pyridin-3-onyl.

Especially preferred compounds include those of formula (I) in which R⁴ denotes a mono- or bicyclic heteroaryl group optionally containing 1 to 3 nitrogen atoms and/or 1 to 2 oxygen atoms and/or 1 to 2 sulfur atoms and/or 1 to 2 keto groups and optionally substituted by one or more groups selected from (C₁-C₅) alkyl groups (which may optionally be substituted by 1 to 3 hydroxyl groups or 1 to 3 COOR¹⁰ groups), (C₁-C₅) alkoxy groups, hydroxyl groups, halogen atoms, (C₁-C₃) exoalkylidene groups where this group may be linked to the amine of the ring system at any position and may optionally be hydrogenated at one or more positions.

A particularly preferred embodiment of the present invention includes compounds of general formula (I) according to Claims 1 through 5, in which R⁴ denotes a bicyclic heteroaryl group, optionally containing 1 to 2 nitrogen and/or 1 keto group and optionally substituted one or more groups selected from (C₁-C₅) alkyl groups, hydroxyl groups, halogen atoms, where this group may be linked to the amine of the ring system at any position and may optionally be hydrogenated at one or more positions.

A preferred object of the present invention is compounds of general formula (I) according to Claims 1 through 5 wherein R⁴ denotes a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group optionally substituted by C₁-C₅ alkyl, halogen, hydroxy, C₁-C₅ alkoxy, keto or (C₁-C₃) exoalkylidene.

A preferred embodiment of the present invention includes compounds of general formula (I) according to Claims 1 through 5 wherein R⁴ denotes a phenyl or naphthyl, phthalidyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group optionally substituted by C₁-C₅ alkyl, halogen, hydroxy or C₁-C₅ alkoxy.

An especially preferred embodiment of the present invention includes compounds of general formula (I) wherein R⁴ denotes a quinolinyl, quinazolinyl, phthalazinonyl, quinolonyl group optionally substituted with C₁-C₃ alkyl, halogen or hydroxy.

When it is a heteroarylalkyl group R⁵, this may optionally also be understood to include a partially hydrogenated heteroaryl group as described above linked to the basic structure by a C₁-C₈ alkyl group which may be linear or branched.

The term heteroarylalkenyl group is understood to refer to a heteroaryl group, optionally also partially hydrogenated, as described above, which may be linked to the basic structure by a C₂-C₈ alkenyl group with may be linear or branched. Another embodiment of the present invention includes compounds of general formula (I) according to Claims 1 through 5 wherein R⁵ denotes a (C₁-C₅) alkyl group or an optionally partially or completely fluorinated (C₁-C₅) alkyl group, a (C₃-C₇) cycloalkyl group, a (C₃-C₇) cycloalkyl (C₁-C₈) alkyl group, (C₃-C₇) cycloalkyl (C₂-C₈) alkenyl group, a heterocyclyl group, a heterocyclyl (C₁-C₈) alkyl group, heterocyclyl (C₂-C₈) alkenyl group, an aryl group, an aryl (C₁-C₈) alkyl group or an aryl (C₂-C₈) alkenyl group.

A special embodiment of the present invention includes compounds of general formula (I) according to Claims 1 through 5 wherein R⁵ denotes a (C₁-C₅) alkyl group or an optionally partially or completely fluorinated (C₁-C₅) alkyl group, an aryl group, an aryl (C₁-C₈) alkyl group, an aryl (C₂-C₈) alkenyl group, an (C₃-C₇) cycloalkyl group, a (C₃-C₇) cycloalkyl (C₁-C₈) alkyl group or a (C₃-C₇) cycloalkyl (C₂-C₈) alkenyl group.

Another embodiment of the present invention includes compounds of general formula (I) according to Claims 1 through 5 wherein R⁵ denotes a (C₁-C₃) alkyl group or an optionally partially or completely fluorinated (C₁-C₃) alkyl group. Compounds of general formula (I) according to Claims 1 through 5 wherein R⁵ denotes a completely fluorinated (C₁-C₃) alkyl group, most especially a CF₃ group are especially preferred.

Another embodiment of the present invention includes compounds of formula (I) wherein R⁴ denotes a C₁-C₁₀ alkyl group, optionally substituted by 1 to 3 hydroxyl groups, halogen atoms, an optionally substituted phenyl group, a mono- or bicyclic heteroaryl group containing 1- to 4 nitrogen atoms and/or 1 to 2 oxygen atoms and/or 1 to 2 sulfur atoms and optionally substituted by 1 to 2 keto groups, 1 to 2 (C₁-C₅) alkyl groups, 1 to 2 (C₁-C₅) alkoxy groups, 1 to 3 halogen atoms, 1 to 2 (C₁-C₃) exoalkylidene groups where these groups may be attached to the nitrogen atom at any position and may optionally be hydrogenated at one or more positions.

The inventive compounds of general formula (I) may be in the form of stereoisomers due to the presence of asymmetry centers. The present invention relates to all possible stereoisomers (e.g., RRR, RRS, RSR, RSS, SRR, SRS, SSR, SSS), both as racemates and as diastereomer mixtures as well as in enantiomer-pure and diastereomer-pure form. The enantiomers and diastereomers can be obtained by methods with which those skilled in the art are familiar, e.g., by chromatography of the racemate and/or the diastereomer mixture on a chiral fixed phase.

The inventive compounds may also be in the form of salts with physiologically compatible anions, e.g., in the form of the hydrochloride, sulfate, nitrate, phosphate, pivalate, maleate, fumarate, tartrate, benzoate, mesylate, citrate or succinate.

The inventive compounds may also be in the form of ethers or esters on the hydroxyl group, which can be synthesized by methods with which those skilled in the art are familiar.

Examples of the ethers include methyl ether, ethyl ether, propyl ether, isopropyl ether, methoxymethyl ether, ethoxymethyl ether and ethoxyethyl ether.

The esters are derived from organic or inorganic acids. The organic acids may be cyclic, linear or branched, saturated or unsaturated, carbocyclic or heterocyclic, substituted or unsubstituted. The radicals are preferably derived from C₁-C₉ carboxylic acids. Examples of acids suitable for esterification include sulfuric acid, formic acid, acetic acid, propionic acid, butanoic acid, pentanoic acid and pivalic acid.

Examples of inorganic acids include H₂SO₄, H₂SO₃, H₂SO₂ and H₃PO₄.

The inventive compounds are synthesized:
a) By converting the cyclic styrenes of general formula (III), synthesized by methods with which those skilled in the art are familiar (e.g., J. Chem. Soc., Perkin Trans. 1 (1999), pp. 2911-2922; Org. Left. 6 (2004), pp. 3047-3050) from the corresponding chromanones, thiochromanones, cyclic ketones or cyclic amino ketones, the conversion being accomplished by an optionally enantioselectively guided En reaction with optionally chiral Lewis acids to yield compounds of general formula (IV). By reduction and amination, the imine (V) is synthesized by methods with which those skilled in the art are familiar 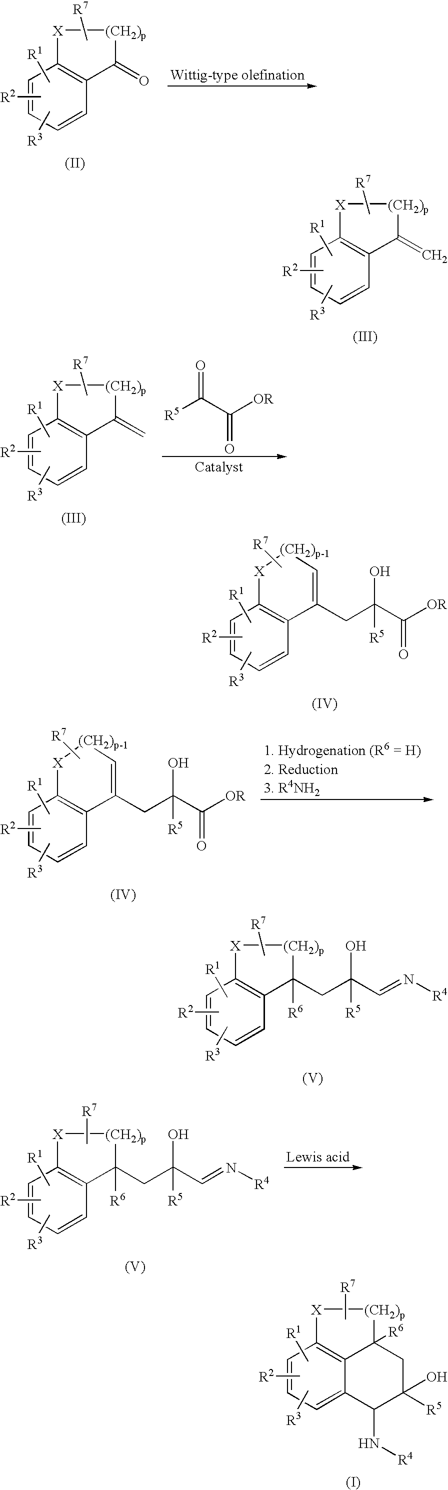
the imine is then cyclized either without further reagent or by adding organic or inorganic acids or Lewis acids at temperatures in the range of −70° C. to +80° C. (preferably in the range of −30° C. to +80° C.) to form compounds of general formula (I). Lewis acid in the sense of the present invention is understood to include all Lewis acids with which those skilled in the art are familiar, e.g., TiCl₄, Ti(OR³)₄, TiCl₂(OR³)₂, TiBr₂(OR³)₂, PdCl₄, Pd(OR³)₄, PdCl₂(OR³)₂, PdBr₂(OR³)₂, ZnCl₂, ZnBr₂, SnCl₄, AICl₃, AlBr₃, AlEtCl₂, AlMe₂Cl, BBR₃, BCl₃, Bl₃, BF₃, BBrMe₂, Cu salts, e.g., Cu(OTf)₂, CuCl₂, CuBr₂, Yb(OTf)₃, preferably BBr₃.

A special embodiment of the present invention includes amines R⁴NH₂, 5-amino-7-fluoro-2-methylquinazoline, 5-amino-8-fluoro-2-methylquinazoline and 5-amino-7,8-difluoro-2-methylquinazoline.
b) Precursors of the type of general formula (VI) can be synthesized by known procedures (J. Med. Chem. 44 (2001), pp. 1085-1098). According to methods with which those skilled in the art are familiar, these compounds can be converted to compounds of general formula (VII) by reduction of the double bond, e.g., with hydrogen on palladium/carbon, reduction of ester, e.g., with lithium aluminum hydride and oxidation of the resulting alcohols.

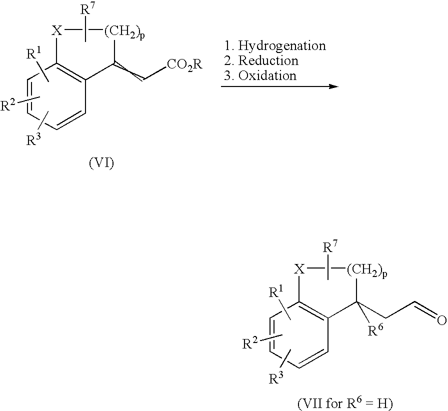

Compounds of type (VII) can be reacted, e.g., with a compound of the general formula C_{q}F_{2q+1}—Si(CH₃)₃ where q=1, 2, 3 or 4, in the presence of a catalyst or with an alkyl metal compound of type R⁵A where A denotes magnesium halogen or lithium, e.g., a Grignard reagent or a lithium alkyl to form a compound of formula (VIII). Fluoride salts or basic compounds such as alkali carbonates may be used as the catalyst (J. Am. Chem. Soc. 111 (1989), p. 393).

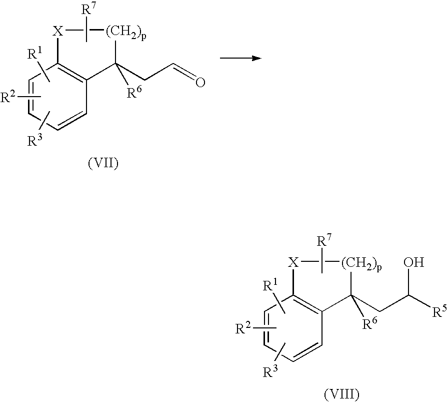

Novel compounds of the type of formula (IX) can be obtained by oxidation of compounds of the general structure (VIII) according to methods known in the literature (J. Org. Chem. 54 (1989), pp. 661-668).

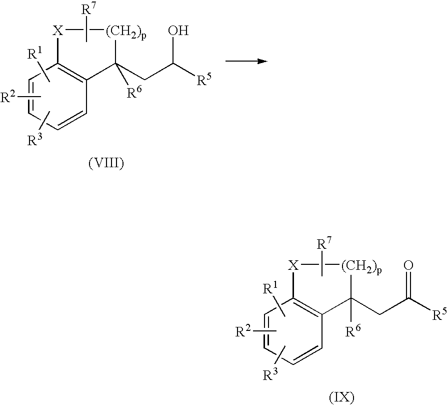

Compounds of general formula (X) can be obtained by reaction of compounds of general formula (IX), e.g., with TMSCN or MCN, where M denotes a metal, e.g., sodium, potassium or copper (J. Med. Chem. 46 (2003), pp. 2494-2501). R¹¹ may optionally be a hydrogen atom or a trialkylsilyl group.

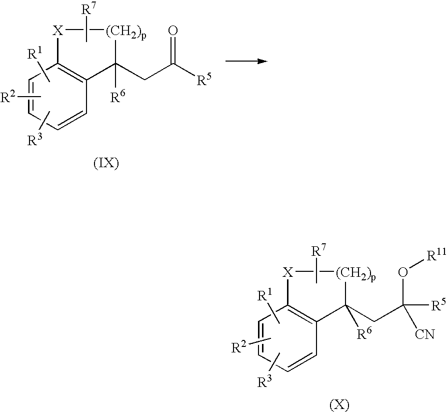

Compounds of general formula (XI) can be synthesized by reaction of compounds of general formula (X) with a suitable reducing agent, e.g., diisobutylaluminum hydride.

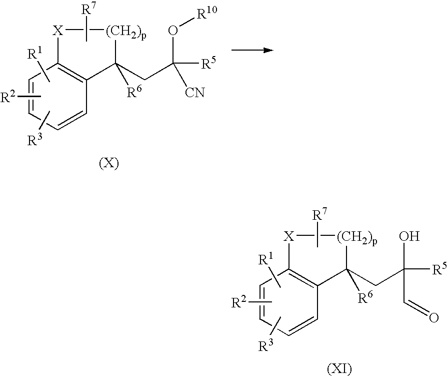

These novel compounds of general formula (XI) may then be converted to compounds of general formula (I) by methods similar to the synthesis process a).
c) Compounds of general formula (VII) [sic; (VI)] can be converted to compounds of formula (VII) by reaction with an alkyl metal compound, e.g., a Grignard reagent or a lithium alkyl in the presence of a suitable copper salt or mixed copper organyls (e.g., Tetrahedron Lett. 31 (1990), pp. 7425-7428; J. Organomet. Chem. 502 (1995), pp. C5-C7) and then reduction, e.g., with diisobutylaluminum hydride, and then the compound of formula (VII) can then be converted to the compounds of general formula (I) by a process similar to synthesis process b). 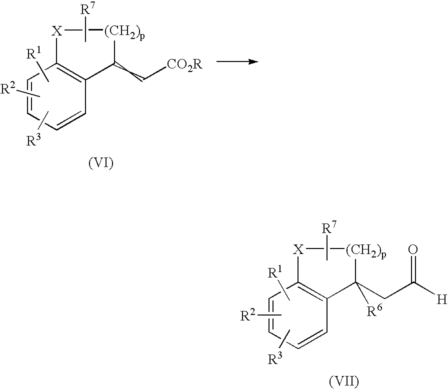
d) Compounds of general formula (IX) may be reacted, e.g., with alkenyl metal organyls R¹²R¹³(C)═CH-A, where A is magnesium halogen or lithium and R¹², R¹³ denote hydrogen or C₁-C₆ alkyl such as vinyl magnesium Grignard compounds. This yields compounds of general formula (XII). Compounds of general formula (X) can be obtained by oxidizing the double bond, e.g., with ozone or with transition metal oxides, e.g., osmium tetraoxide with subsequent cleavage using a suitable oxidizing agent such as sodium periodate.

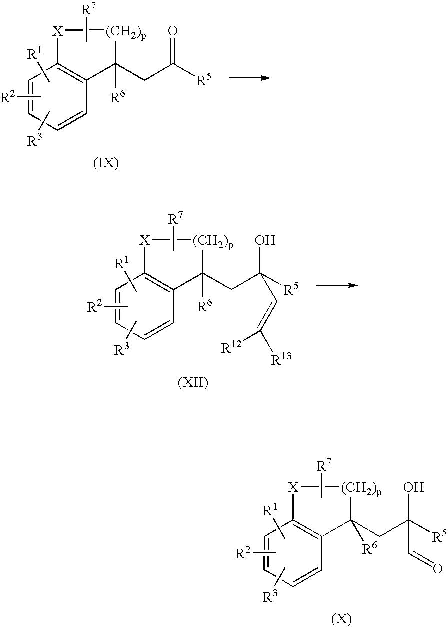

An embodiment of the present invention includes the intermediates involved in the processes described above, in particular the compounds of formulas (III), (IV) and (V) where the radicals have the meanings given in Claim 1 and the process steps for synthesis of same.

The binding of substances to the glucocorticoid receptor (GR) and other steroid hormone receptors (mineral corticoid receptor (MR), progesterone receptor (PR) and androgen receptor (AR)) is tested with the help of recombinant receptors. Cytosol preparations of Sf9 cells infected with recombinant baculoviruses that code for GR are used for binding tests. In comparison with the reference substance [³H]-dexamethasone, these substances have a high affinity for GR. An IC₅₀ (GR)=20 nM and IC₅₀ (PR)>1 μM have been obtained for the compound from Example 2.

The inhibition of transcription of cytokines, adhesion molecules, enzymes and other pro-inflammatory factors is thought to be as an essential molecular mechanism for the anti-inflammatory action of glucocorticoids. This inhibition is induced by an interaction of GR with other transcription factors, e.g., AP-1 and NF-kappa-B (for a review, see Cato, A. C. B. and Wade, E., BioEssays 18, 371-378, 1996).

The inventive compounds of general formula (I) inhibit the secretion of cytokine IL-8 triggered by lipopolysaccharide (LPS) in the human monocyte cell line THP-1. The concentration of cytokines was determined in the supernatant using commercially available ELISA kits. The compound from example 2 shows inhibition of IC₅₀ (IL8)=37 nM with a 70% efficiency with respect to [³H]-dexamethasone as the standard.

The anti-inflammatory effect of the compounds of general formula (I) has been tested in animal experiments by testing in the croton oil-induced inflammation in the rat and the mouse (J. Exp. Med. (1995) 182, 99-108). To do so, croton oil in ethanolic solution was administered topically to the ears of the animals. The test substances were also administered topically or systemically either simultaneously or 2 hours before the croton oil. After 16 to 24 hours, the weight of the ear was determined as a measure of the inflammatory edema, the peroxidase activity was determined as a measure of the migration of granulocytes and the elastase activity was determined as a measure of the migration of neutrophilic granulocytes. In this test, compounds of general formula (I) inhibit the three inflammation parameters mentioned above after both topical and systemic administration.

One of the most common adverse effects of a glucocorticoid treatment is the so-called “steroid diabetes” (see Hatz, H. J., Glucocorticoide: Immunologische Grundlagen, Pharmakologie und Therapierichtlinien [Glucocorticoids: Immunological Principles, Pharmacology and Treatment Guidelines], Wissenschaftliche Verlagsgesellschaft mbH [Scientific Publishing Co.], Stuttgart, 1998). The cause for this is stimulation of the gluconeogenesis in the liver by induction of the responsible enzymes and by free amino acids formed by degradation of proteins (catabolic effect of glucocorticoids). A key enzyme of the catabolic metabolism in the liver is tyrosine amino transferase (TAT). The activity of this enzyme can be determined photometrically from liver homogenates and is a good measure of the adverse metabolic effects of glucocorticoids. To measure TAT induction, experimental animals were sacrificed 8 hours after administration of the test substances, the liver was removed and TAT activity in the homogenate was measured. In doses in which these compounds have an anti-inflammatory activity, the compounds of general formula (I) do not induce tyrosine amino transferase in this test or do so only to a minor extent.

Because of their anti-inflammatory and also anti-allergic immunosuppressant and anti-proliferative effects, the inventive compounds of general formula (I) may be used as medications to treat or prevent the following disease states in mammals and humans, where the term “DISEASE” is used for the following indications:
(i) Lung diseases associated with inflammatory, allergic and/or proliferative processes:
   chronic obstructive lung diseases of any genesis, especially bronchial asthma
   bronchitis of varying genesis
   all forms of restrictive lung disease, especially allergic alveolitis
   all forms of pulmonary edema, especially toxic pulmonary edema
   sarcoidoses and granulomatoses, in particular Boeck's disease
(ii) Rheumatic diseases, autoimmune diseases, joint diseases associated with inflammatory, allergic and/or proliferative processes:
   all forms of rheumatic diseases, in particular rheumatoid arthritis, acute rheumatic fever, polymyalgia rheumatica
   reactive arthritis
   inflammatory soft tissue diseases of other genesis
   arthritic symptoms of degenerative joint diseases (arthroses)
   traumatic arthritis
   collagenoses of any genesis, e.g., systemic lupus erythematosus, sclerodermia, polymyositis, dermatomyositis, Sjörgen's syndrome, Still syndrome, Felty syndrome
(iii) Allergies associated with inflammatory and/or proliferative processes:
   all forms of allergic reactions, e.g., Quincke's edema, hay fever, insect bites, allergic reactions to drugs, blood derivatives, contrast media, etc., anaphylactic shock, urticaria, contact dermatitis
(iv) Vasculitis
   panarteriitis nodosa, arteriitis temporalis, erythema nodosum
(v) Dermatologic diseases associated with inflammatory, allergic and/or proliferative processes:
   atopic dermatitis (especially in children)
   psoriasis
   pityriasis rubra pilaris
   erythematous diseases caused by various noxious effects, e.g., radiation, chemicals, burns, etc.
   bullous dermatoses
   diseases of the lichenoid group
   pruritus (e.g., of allergic genesis)
   seborrheic eczema
   rosacea
   pemphigus vulgaris
   exudative erythema multiforme
   balanitis
   vulvitis
   hair loss, such as alopecia areata
   cutaneous T-cell lymphoma
(vi) Renal diseases associated with inflammatory, allergic and/or proliferative processes:
   nephrotic syndrome
   all nephritis conditions
(vii) Hepatic diseases associated with inflammatory allergic and/or proliferative processes:
   acute hepatolysis
   acute hepatitis of varying genesis, e.g., viral, toxic or drug induced
   chronic aggressive and/or chronic intermittent hepatitis
(viii) Gastrointestinal diseases associated with inflammatory, allergic and/or proliferative processes:
   regional enteritis (Crohn's disease)
   ulcerative-colitis
   gastritis
   reflux esophagitis
   gastroenteritis of other genesis, e.g., native sprue
(ix) Proctologic diseases associated with inflammatory, allergic and/or proliferative processes:
   anal eczema
   fissures
   hemorrhoids
   idiopathic proctitis
(x) Ophthalmic diseases associated with inflammatory, allergic and/or proliferative processes:
   allergic keratitis, uveitis, iritis
   conjunctivitis
   blepharitis
   neuritis of the optic nerve
   chorioditis
   sympathetic ophthalmia
(xi) Disease of the ear, neck and throat area associated with inflammatory, allergic and/or proliferative processes:
   allergic rhinitis, hay fever
   otitis externa, e.g., due to contact eczema, infection, etc.
   otitis media
(xii) Neurologic diseases associated with inflammatory, allergic and/or proliferative processes:
   cerebral edema, especially due to a tumor
   multiple sclerosis
   acute encephalomyelitis
   meningitis
   various forms of seizures, e.g., BNS seizures
(xiii) Blood diseases associated with inflammatory, allergic and/or proliferative processes:
   acquired hemolytic anemia
   idiopathic thrombocytopenia
(xiv) Tumor diseases associated with inflammatory, allergic and/or proliferative processes:
   acute lymphatic leukemia
   malignant lymphomas
   lymphogranulomatoses
   lymphosarcoma
   extensive metastases, especially with breast cancer, bronchial cancer and prostate cancer
(xv) Endocrine diseases associated with inflammatory allergic and/or proliferative processes:
   endocrine orbitopathy
   thyrotoxic crisis
   De Quervain's thyroiditis
   Hashimoto's thyroiditis
   Basedow's disease
(xvi) Organ and tissue transplants, graft versus host disease
(xvii) Severe shock states, e.g., anaphylactic shock, systemic inflammatory response syndrome (SIRS)
(xviii) Replacement therapy in:
   congenital primary adrenal insufficiency, e.g., congenital adrenogenital syndrome
   acquired primary adrenal insufficiency, e.g., Addison's disease, autoimmune adrenalitis, post-infectious tumors, metastases, etc.
   congenital secondary adrenal insufficiency, e.g., congenital hypopituitarism
   acquired secondary adrenal insufficiency, e.g., post-infectious tumors, etc.
(xix) Emesis associated with inflammatory, allergic and/or proliferative processes:
   e.g., in combination with a 5-HT-3 antagonist in cytostic [sic; cytostatic]induced vomiting
(xx) Pain of inflammatory genesis, e.g., lumbago.

The invention also relates to combination therapies or combined formulations in which a glucocorticoid receptor (GR), agonist of formula (I) or a pharmaceutically acceptable salt thereof or a pharmaceutical composition containing a GR agonist of formula (I) or a pharmaceutically acceptable salt thereof is administered either concurrently (optionally in the same composition) or in succession together with one or more pharmaceutical drugs for treatment of one of the aforementioned disease states. For treatment of rheumatoid arthritis, osteoarthritis, COPD (chronic obstructive pulmonary disease), asthma or allergic rhinitis, for example, a GR agonist of the present invention may be combined with one or more pharmaceutical drugs for treatment of such a state. If such a combination is administered by inhalation, the pharmaceutical drug to be combined with it may be selected from the following list:
a PDE4 inhibitor including an inhibitor of the iso form PDE4D;
a selective β₂-adrenoreceptor agonist, e.g., metaproterenol, isoproterenol, isoprenaline, albuterol, salbutamol, formoterol, salmeterol, terbutaline, orciprenaline, bitoiterol mesylate, pirbuterol or indacaterol;
a muscarine receptor antagonist (e.g., an M1, M2 or M3 antagonist, e.g., a selective M3 antagonist) such as ipratropium bromide, tiotropium bromide, oxitropium bromide, pirenzepine or telenzepine;
a modulator of the chemokine receptor function (e.g., a CCR1 receptor antagonist); or
an inhibitor of the p38 kinase function.

For another embodiment of the present invention, a combination with a GR agonist of formula (I) or a pharmaceutically acceptable salt thereof is used for treatment of COPD, asthma or allergic rhinitis and may be administered by inhalation or orally in combination with xanthine (e.g., aminophylline or theophylline) which may also be administered orally or by inhalation.

In addition, the inventive compounds of general formula (I) may be used for treatment and prevention of other disease states not mentioned above for which synthetic glucocorticoids are used today (see Hatz, H. J., Glucocorticoide: Immunologische Grundlagen, Pharmakologie und Therapierichtlinien [Glucocorticoids: Immunological Principles, Pharmacology and Treatment Guidelines], Wissenschaftliche Verlagsgesellschaft mbH [Scientific Publishing Co.], Stuttgart, 1998).

All the indications (i) through (xx) listed above are described in detail in Hatz, H. J., Glucocorticoide: Immunologische Grundlagen, Pharmakologie und Therapierichtlinien [Glucocorticoids: Immunological Principles, Pharmacology and Treatment Guidelines], Wissenschaftliche Verlagsgesellschaft mbH [Scientific Publishing Co.], Stuttgart, 1998.

The suitable dose to achieve the therapeutic effects in the aforementioned disease conditions varies and depends, for example, on the potency of the compounds of general formula (I), the host, how it is administered and the type and severity of the condition to be treated as well as whether it is being used as a prophylactic or therapeutic agent.

The present invention also provides
(i) Use of one of the inventive compounds according to formula (I) or a combination thereof for preparing a medication for treatment of a DISEASE;
(ii) A method for treatment of a DISEASE, said method including administration of a quantity of the compound according to this invention, where said quantity of said compound suppresses the illness and said quantity of said compound is administered to a patient needing such a medication;
(iii) A pharmaceutical composition for treatment of a DISEASE which includes treatment of [sic; with] one of the inventive compounds or a mixture thereof and at least one pharmaceutical excipient and/or vehicle.

In general, satisfactory results are to be expected in animals when the daily dose is in a range of 1 μg to 100,000 μg of the inventive compound per kilogram of body weight. A dose of 10 to 30,000 μg per kg body weight is preferred, but even more preferred is a dose of 10 to 10,000 μg per kg body weight. For example, this dose may expediently be administered several times daily. For treatment of acute shock (e.g., anaphylactic shock) single doses, which are definitely greater than the aforementioned doses, may be administered.

The pharmaceutical preparations based on the novel compounds are formulated in a known way (e.g., by processing the active ingredient with the vehicle substances, fillers, disintegrants, binders, humectants, lubricants, absorbents, diluents, taste correctors, coloring agents, etc. conventionally used in pharmaceutical technology and are converted to the desired form of administration. Reference is made here to Remington's Pharmaceutical Science, 15^{th} edition, Mack Publishing Company, East Pennsylvania (1980).

Tablets, capsules, pills, coated pills, powders, granules, lozenges, suspensions, emulsions and solutions may be used for oral administration.

Injection and infusion preparations are possible for parenteral administration.

Suitably prepared crystal suspensions may be used for intra-articular injection.

For intramuscular injection, aqueous and oil-based injection solutions or suspensions and the corresponding depot preparations may be used.

For rectal administration, the novel compounds may be used in the form of suppositories, capsules, solutions (e.g., in the form of enemas) and ointments for both systemic and topical treatment.

For pulmonary administration of the novel compounds, they may be used in the form of aerosols and inhalates.

For topical administration to the eyes, external auditory canal, middle ear, nasal sinuses and paranasal sinuses, the novel compounds may be used in the form of drops, ointments and tinctures in corresponding pharmaceutical preparations.

For topical administration, possible formulations include gels, ointments, fat-based ointments, creams, pastes, powders, milks and tinctures. The dosage of the compounds of general formula (I) in these preparations should be 0.01% to 20% to achieve an adequate pharmacological effect.

This invention also includes the inventive compounds of general formula (I) as therapeutic active ingredients. In addition, the present invention includes the inventive compounds of, general formula (I) as therapeutic active ingredients together with pharmaceutically tolerable and acceptable excipients and vehicles belong to the present invention.

The present invention also includes a pharmaceutical composition containing one of the pharmaceutically active inventive compounds or a mixture thereof or their pharmaceutically tolerable salts and pharmaceutically tolerable excipients and vehicles.

### EXPERIMENTAL PART

### Example 1

### 6-[(7-Fluoro-2-methylquinazolin-5-yl)amino]-5-(trifluoromethyl)-2,3,3a,4,5,6-hexahydrobenzo[de]chromen-5-ol

### 3-(2-Chroman-4-yl)-2-hydroxy-2-(trifluoromethyl)propanal

20 g (307 mmol) zinc dust and 710 mg (2.5 mmol) lead(II) chloride are suspended in 200 mL THF, and 11.2 mL (100 mmol) dibromomethane is added at room temperature. Stirring is continued for 60 minutes more and then 33 mL (33 mmol) of a 1M titanium(IV) chloride solution in dichloromethane is added by drops over 40 minutes while cooling with ice. After 1 hour, 4.4 g (30 mmol) chroman-4-one is added in solid form in several portions while the reaction temperature rises to 30° C. Stirring is continued for 3 more hours at room temperature. The mixture is diluted with diethyl ether and the reaction mixture is cautiously added to a mixture of 4M hydrochloric acid and ice. The phases are separated, extracted with ether, washed with water, dried over sodium sulfate and the solvent is removed. The raw product is purified by column chromatography on silica gel (hexane/isopropyl ether 0-20%), yielding 2.75 g 4-methylenechroman.

0.60 mL (0.3 mmol) of a 0.5M titanium tetraisopropylate solution in toluene is added to 170 mg (0.60 mmol) 1,1′-bi-2-naphthol and the red solution is stirred for 2 hours at room temperature. Then 1.0 g (6.8 mmol) 4-methylenechroman and 2.3 [sic; no units given] (13.6 mmol) ethyl trifluoropyruvate are added and the mixture is heated for 2 hours at 110° C. After cooling, the mixture is purified immediately by column chromatography on silica gel (hexane/ethyl acetate 20%), yielding 1.15 g 3-(2H-chromen-4-yl)-2-hydroxy-2-(trifluoromethyl)propionic ethyl ester. 100 mg (0.32 mmol) 3-(2H-chromen-4-yl)-2-hydroxy-2-(trifluoromethyl)propionic acid ethyl ester is dissolved in 10 mL methanol and then 20 mg palladium on carbon 10% is added. The reaction mixture is agitated under a hydrogen atmosphere for 5 hours. Then the reaction mixture is filtered through Celite, rewashed with ethyl acetate and the solvent is removed in vacuo. The residue is placed in 10 mL diethyl ether and cooled to −5° C. Over a period of 10 minutes, 78 mg (2.0 mmol)

lithium aluminum hydride in solid form is added by portions. The mixture is stirred for 2 hours at room temperature and then poured into saturated ammonium chloride solution. The solution is filtered through Celite, washing thoroughly with ethyl acetate. The phases of the filtrate are separated and extracted again with ethyl acetate, then washed with saturated sodium chloride solution, dried over sodium sulfate and the solvent is removed in vacuo. Chromatographic purification on silica gel (hexane/ethyl acetate 0-15%) yields 50 mg 3-(2H-chromen-4-yl)-2-hydroxy-2-(trifluoromethyl)propanal as a mixture of two diastereomers. ¹H-NMR (300 MHz, CDCl₃); δ=1.66 (m, 0.5H), 1.95-2.37 (m, 2.5H), 2.49 (dd, 0.5H), 2.54 (dd, 1H), 2.79 (m, 0.5H), 3.14 (m, 0.5H), 3.89 (s, 0.5H), 3.99 (s, 0.5H), 4.09-4.22 (m, 2H), 6.78-6.95 (m, 1H), 7.08-7.19 (m, 2H), 9.74 (s, 1H).

### 5-Amino-7-fluoro-2-methylquinazoline

17 g (70.5 mmol) 3,6-difluoro-2-N-pivaloylaminobenzaldehyde (L. Florvall, I. Fagervall, L. G. Larsson, S. B. Ross, Eur. J. Med. Chem. 34 (1999), 137-151), 9.2 g acetamidine hydrochloride, 13.4 g potassium carbonate and 10.4 g molecular sieve (4A) are added to 70 mL butyronitrile. The mixture is heated with vigorous agitation for 17 hours at 145° C. and then the solvent is removed in vacuo. After chromatography of the residue on silica gel with hexane/ethyl acetate (0 to 70%), 4.5 g 7-fluoro-5-N-pivaloylamino-2-methylquinazoline is obtained. Next 1 g (3.82 mmol) 7-fluoro-5-N-pivaloylamino-2-methylquinazoline is dissolved in 74 mL toluene and cooled to −70° C. Over a period of 30 minutes, 9.5 mL (11.4 mmol) of a 1.2M diisobutylaluminum hydride solution is added by drops to toluene. The reaction mixture is allowed to warm up to −40° C. and agitated for 4 hours at −40° C. Then water is added slowly and agitated for 30 minutes at room temperature until forming a precipitant, which is then removed by filtration through Celite. The phases are separated, washed with saturated sodium chloride solution and dried over sodium sulfate. After chromatography on silica gel with hexane-ethyl acetate (0-100%), 64 mg of the product is obtained.

¹H-NMR (300 MHz, CDCl₃); δ=2.83 (s, 3H), 4.67 (br, 2H), 6.50 (dd, 1H), 6.93 (dd, 1H), 9.23 (s, 1H).

50 mg (0.18 mmol) 3-(2H-chroman-4-yl)-2-hydroxy-2-(trifluoromethyl)propanol is dissolved in 5 mL toluene together with 20 mg (0.12 mmol) 5-amino-7-fluoro-2-methylquinazoline and then 0.15 mL titanium tetraethylate is added. The mixture is heated to 100° C. for 2 hours. After cooling, the mixture is poured onto water and agitated vigorously. The suspension is filtered through Celite, washing again thoroughly with ethyl acetate. The phases of the filtrate are separated and extraction with ethyl acetate is performed again. The product is dried over sodium sulfate and the solvent is removed in vacuo, yielding 3-(chroman-4-yl)-1-[(7-fluoro-2-methylquinazolin-5-yl)imino]-2-(trifluoromethyl)propan-2-ol as a raw product. The imine is placed in 4 mL dichoromethane and cooled to −78° C. Within 5 minutes, 1.5 mL (1.5 mmol) of a 1M titanium tetrachloride solution in dichloromethane is added; after 20 minutes the cooling bath is removed and then after another 20 minutes, the solution, now heated to room temperature, is poured onto a mixture of ice and saturated sodium bicarbonate solution, then agitating vigorously for 10 minutes. The mixture is extracted with ethyl acetate, washed with saturated sodium chloride solution and dried over sodium sulfate. Concentrating and chromatography on silica gel (hexane/ethyl acetate 50-100%) yield 9 mg of the desired product.

¹H-NMR (300 MHz, CDCl₃); δ8=1.78-1.91 (m, 2H), 2.06 (dd, 1H), 2.48 (dd, 1H), 3.34 (m, 1H), 4.26 (ddd, 1H), 4.50 (ddd, 1H), 5.13 (d, 1H), 5.79 (d, 1H), 6.65 (dd, 1H), 6.74 (d, 1H), 6.82 (d, 1H), 6.94 (dd, 1H), 7.07 (t, 1H), 9.19 (s, 1H).

### Example 2

### 9-Fluoro-6-[(2-methylquinolin-5-yl)amino]-5-(trifluoromethyl)-2,3,3a,4,5,6-hexahydrobenzo[de]chromen-5-ol

### 3-(8-Fluorochroman-4-yl)-2-hydroxy-2-(trifluoromethyl)propanal

33.2 g (296 mmol) 2-fluorophenol and 18.4 mL (281 mmol) acrylonitrile are agitated together with 5.0 g (29.6 mmol) benzyltrimethylammonium hydroxide for 4 days at 80° C., then combined with ice at room temperature and 2N hydrochloric acid is added. The mixture is agitated for 10 minutes, extracted with ethyl acetate, washed with saturated sodium bicarbonate and sodium chloride solution and dried over

sodium sulfate and then the solvent is removed in vacuo. The separation by column chromatography on silica gel (hexane/ethyl acetate 5-50%) yields 6.4 g 3-(2-fluoro-phenoxy)propionitrile. 6.4 g (38.8 mmol) 3-(2-fluorophenoxy)propionitrile is refluxed for 2 hours in 38.6 mL concentrated hydrochloric acid. The mixture is diluted with ice water at room temperature and agitated for 10 minutes, then extracted with ethyl acetate, washed with saturated sodium chloride solution and dried over sodium sulfate. After removing the solvent in vacuo, 6.5 g 3-(2-fluorophenoxy)propionic acid is obtained. 6.5 g (35.6 mmol) 3-(2-fluorophenoxy)propionic acid is added to 39 g polyphosphoric acid and agitated for 4 hours at 70° C. After cooling overnight, the mixture is poured onto ice water, extracted with ethyl acetate, washed with saturated sodium bicarbonate and sodium chloride solution and dried over sodium sulfate. Removing the solvent in vacuo yields 5.5 g 8-fluorochroman-4-one as a crystalline solid.

29.8 g (456 mmol) zinc dust and 710 mg (2.5 mmol) lead(II) chloride are suspended in 450 mL THF and 28.6 mL (253 mmol) dibromomethane is added at room temperature. The mixture is stirred for 60 minutes more at 50.7 mL (50.7 mmol) of a 1M titanium(IV) chloride solution in dichloromethane is added by drops over a period of 40 minutes while cooling with ice. After 1 hour, 8.4 g (50.7 mmol) 8-fluorochroman-4-one is added by drops to 500 mL THF at room temperature. This mixture is agitated for another 18 hours at room temperature, diluted with diethyl ether and the reaction mixture is cautiously added to a mixture of 4M hydrochloric acid and ice. The phases are separated, extracted with diethyl ether, washed with water, dried over sodium sulfate and the solvent is removed. The raw product is purified by column chromatography on silica gel (hexane/isopropyl ether 0-20%), yielding 0.81 g 8-fluoro-4-methylenechroman. To 281 mg (0.98 mmol) 1,1′-bi-2-naphthol is added 0.98 mL (0.49 mmol) of a 0.5M titanium tetraisopropylate solution in toluene, and the red solution is agitated for 2 hours at room temperature. 0.81 g (4.9 mmol) 8-fluoro-4-methylenechroman and 1.21 mL (9.8 mmol) ethyl trifluoropyruvate are added and the mixture is heated for 3 hour at 120° C. After cooling, the mixture is purified immediately by column chromatography on silica gel (hexane/ethyl acetate 0-20%), yielding 0.69 g 3-(8-fluoro-2H-chromen-4-yl)-2-hydroxy-2-(trifluoromethyl)propionic acid ethyl ester. 400 mg (1.2 mmol) 3-(8-fluoro-2H-chromen-4-yl)-2-hydroxy-2-(trifluoromethyl)propionic acid ethyl ester is dissolved in 12 mL methanol and 40 mg palladium on carbon 10% is added. The reaction mixture is agitated under a hydrogen atmosphere for 40 minutes. Then it is filtered through Celite, washed with dichloromethane and the solvent is removed in vacuo, yielding 330 mg crude 3-(8-fluorochroman-4-yl)-2-hydroxy-2-(trifluoromethyl)propionic acid ethyl ester, which is cooled to a −5° C. in 8 mL diethyl ether and 2 mL THF and 185 mg (4.9 mmol) lithium aluminum hydride in solid form is added by portions. The mixture is added for 24 hours at room temperature and then poured into water. The phases are separated and extracted several times with ethyl acetate, then washed with saturated sodium chloride solution and dried over sodium sulfate and the solvent is removed in vacuo. Chromatographic separation on silica gel (hexane/ethyl acetate 0-15%) yields 53 mg 3-(8-fluorochroman-4-yl)-2-hydroxy-2-(trifluoromethyl)propanol has a mixture of two diastereomers and 126 mg alcohol.

¹H-NMR (300 MHz, CDCl₃); δ=1.73 (m, 0.5H), 1.99-2.38 (m, 2.5H), 2.47 (dd, 0.5H), 2.53 (dd, 1H), 2.84 (m, 0.5H), 3.16 (m, 0.5H), 3.90 (s, 0.5H), 4.00 (s, 0.5H), 4.18-4.37 (m, 2H), 6.70-6.97 (m, 3H), 9.75 (s, 1H).

By analogy with Example 1, 53 mg (0.18 mmol) 3-(8-fluorochroman-4-yl)-2-hydroxy-2-(trifluoromethyl)propanol is reacted with 30 mg (0.19 mmol) 5-amino-2-methylquinoline to form the corresponding 3-(8-fluorochroman-4-yl)-1-[(2-methylquinolin-5-yl)imino]-2-(trifluoromethyl)propan-2-ol. The imine is placed in 3.6 mL dichloromethane and treated at −50° C. with 0.90 mL (0.90 mmol) of a 1M boron tribromide solution. The mixture is allowed to heat up to 0° C. over a period of 60 minutes, and then the solution is poured onto a mixture of ice and saturated sodium bicarbonate solution, then agitated vigorously for 10 minutes, extracted with dichloromethane, washed with saturated sodium chloride solution and dried over sodium sulfate. After concentrating and chromatography on silica gel (hexane/2-propanol 10-20%), 25 mg of the desired product is obtained as a mixture of two diastereomers.

Diastereomer 1

¹H-NMR (300 MHz, CDCl₃); δ=1.70-1.86 (m, 2H), 2.10 (m, 1H), 2.47 (dd, 1H), 2.72 (s, 3H), 3.31 (m, 1H), 4.27 (ddd, 1H), 4.40 (d, 1H), 4.60 (ddd, 1H), 5.20 (d, 1H), 6.61 (d, 1H), 6.81 (dd, 1H), 6.97 (dd, 1H), 7.22 (d, 1H), 7.58 (t, 1H), 7.59 (d, 1H), 7.98 (d, 1H).

Diastereomer 2

¹H-NMR (300 MHz, CDCl₃); δ=1.70-1.86 (m, 2H), 2.10 (dd, 1H), 2.23 (m, 1H), 2.73 (s, 3H), 3.00 (m, 1H), 4.24 (ddd, 1H), 4.54 (ddd, 1H), 4.78 (d, 1H), 5.10 (d, 1H), 6.58 (d, 1H), 6.74 (dd, 1H), 6.89 (dd, 1H), 7.22 (d, 1H), 7.48 (d, 1H), 7.49 (t, 1H), 8.12 (d, 1H).

### Example 3

### 9-Fluoro-6-[(2-methylquinazolin-5-yl)amino]-5-(trifluoromethyl)-2,3,3a,4,5,6-hexahydrobenzo[de]chromen-5-ol

### 5-Amino-2-methylquinazoline

12.7 g (62 mmol) 2-methyl-5-nitro-3H-quinazolin-4-one (M. T. Bogert, V. J. Chambers, J. Org. Chem. 1905, 649-658) and 37.5 g phosphorus pentachloride are heated at reflux for 20 hours in 75 mL phosphoryl chloride. After cooling, the mixture is poured into saturated NaHCO₃ solution and extracted with ethyl acetate. The organic phase is dried and the solvent is removed, yielding 14 g 4-chloro-2-methyl-5-nitroquinazoline, 4.5 g (20.2 mmol) of which is dissolved in 225 mL ethyl acetate and 22.5 mL triethylamine. To this is added 2 g palladium on carbon and the mixture is stirred for 4 hours at standard pressure under a hydrogen atmosphere while cooling with ice. The solution is freed of the catalyst by filtration through Celite, then rewashed with 200 mL ethanol and evaporated. After chromatography on silica gel with ethyl acetate-ethanol (0-10%), 530 mg of the product is obtained.

¹H-NMR (300 MHz, CDCl₃); δ=2.87 (s, 3H), 4.52 (br, 2H), 6.77 (d, 1H), 7.33 (d, 1H), 7.65 (t, 1H), 9.40 (s, 1H).

To 60 mg (0.21 mmol) 3-(8-fluorochroman-4-yl)-2-hydroxy-2-(trifluoromethyl)propanal and 39 mg (0.22 mmol) 5-amino-2-methylquinazoline in 4 mL toluene is added 89 μL (0.42 mmol) titanium tetraethylate and the mixture is heated to 110° C. for 2 hours. After cooling, ethyl acetate is added to saturated ammonium chloride solution and agitated vigorously. The suspension is filtered through Celite, washing again thoroughly with ethyl acetate. The phases of the filtrate are separated and extracted again with ethyl acetate. The resulting crude 3-(8-fluorochroman-4-yl)-1-[(2-methylquinoazolin-5-yl)imino]-2-(trifluoromethyl)propan-2-ol is placed in 2.6 mL dichloromethane and cooled to −50° C. 0.67 mL (0.67 mmol) of a 1M boron tribromide solution in dichloromethane is added by drops over 5 minutes and the cooling bath is removed. After 2 hours the cold solution at a temperature of approximately ⁰° C. is poured onto a mixture of ice and saturated sodium bicarbonate solution and agitated vigorously for 15 minutes, extracted with dichloromethane, washed with saturated sodium chloride solution and dried over sodium sulfate. After concentrating and chromatographic separation on silica gel (hexane/2-propanol 15%), 6 mg of the desired product is obtained.

¹H-NMR (300 MHz, CDCl₃); δ8=1.82-1.92 (m, 2H), 2.10 (dd, 1H), 2.50 (dd, 1H), 2.85 (s, 3H), 3.36 (dddd, 1H), 4.29 (ddd, 1H), 4.62 (ddd, 1H), 5.19 (d, 1H), 5.38 (d, 1H), 6.74 (dd, 1H), 6.87 (dd, 1H), 6.94 (d, 1H), 7.37 (3, 1H), 7.77 (t, 1H), 9.33 (s, 1H).

### Example 4

### {[9-Fluoro-5-hydroxy-5-(trifluoromethyl)-2,3,3a,4,5,6-hexahydrobenzo[de]chromen-6-yl]amino}-2-methylphthalazin-1-one

### 5-Amino-2-methylphthalazin-1-one

### 3-Bromo-4-nitrophthalide

5.37 g 4-nitrophthalide (Tetrahedron Lett. (2001), 42, pp. 1647-50), 8.04 g N-bromosuccinimide and 196 mg benzoyl peroxide are heated at reflux in 80 mL benzotrifluoride under the influence of light until a complete reaction is obtained. The mixture is poured onto water, extracted with dichloromethane, washed repeated with water, dried and the solvent removed in vacuo, yielding 7.24 g 3-bromo-4-nitrophthalide in the form of a solid.

¹H-NMR (300 MHz, CDCl₃); δ=7.26 (s, 1H), 7.88 (t, 1H), 8.3 (d, 1H), 8.56 (d, 1H).

### 5-Nitrophthalazin-1-one

18.25 g hydrazine sulfate and 14.88 g sodium carbonate are agitated for 1 hour in 300 mL DMF at 100° C. Then 7.24 g 3-bromo-4-nitrophthalide is added to 100 mL DMF and agitated for 4 hours more at 100° C., then poured onto water, extracted several times with ethyl acetate and the organic phase washed with water and brine. The product is dried and the solvent is removed in vacuo. After recrystallizing from ethyl acetate, 2.35 g 5-nitrophthalazin-1-one is obtained in the form of solids.

¹H-NMR (300 MHz, DMSO-d₆); δ=8.05 (t, 1H), 8.57-8.66 (m, 2H), 08.73 (s, 1H), 13.13 (bs, 1H).

### 2-Methyl-5-nitrophthalazin-1-one

1.6 g 5-nitrophthalazin-1-one and 2.31 g potassium carbonate are agitated for 10 minutes at room temperature in 60 mL DMF. Then 1.1 mL methyl iodide is added and the mixture is agitated overnight, poured onto water, extracted repeatedly with ethyl acetate and the organic phase is washed with water and brine, then dried and the solvent removed in vacuo, yielding 1.57 g 2-methyl-5-nitrophthalazin-1-one as yellow solids.

¹H-NMR (300 MHz, DMSO-d₆); δ=3.73 (s, 3H), 8.05 (t, 1H), 8.62 (d, 2H), 8.75 (s, 1H).

### 5-Amino-2-methylphthalazin-1-one

1.57 g 2-methyl-5-nitrophthalazin-1-one and 130 mg palladium on activated carbon are suspended in 45 mL ethyl acetate and hydrogenated with hydrogen under normal pressure. The mixture is filtered through diatomaceous earth and the solvent is removed in vacuo, yielding 1.26 g 5-amino-2-methylphthalazin-1-one as yellow solids.

¹H-NMR (300 MHz, CDCl₃); δ=3.81 (s, 3H), 7.0 (d, 1H), 7.5 (t, 1H), 7.8 (dd, 1H), 8.16 (s, 1H).

By analogy with Example 3, 60 mg (0.21 mmol) 3-(8-fluorochroman-4-yl)-2-hydroxy-2-(trifluoromethyl)propanol is reacted with 39 mg (0.22 mmol) 5-amino-2-methylphthalazin-1-one to yield a corresponding {[3-(8-fluorochroman-4-yl)-2-hydroxy-2-(trifluoromethyl)propylidene]amino}-2-methylphthalazin-1-one. The imine is placed in 3.4 mL dichloromethane and treated at −50° C. with 0.83 mL (0.83 mmol) of a 1M boron tribromide solution. The mixture is allowed to heat up to 0° C. for 1 hour and the solution is poured onto a mixture of ice and saturated sodium bicarbonate solution, then agitated vigorously for 15 minutes, extracted with dichloromethane, washed with saturated sodium chloride solution and dried over sodium sulfate. After concentrating and chromatography on silica gel (hexane/2-propanol 15%), 28 mg of the desired product is obtained as a mixture of two diastereomers.

Diastereomer 1

¹H-NMR (300 MHz, CDCl₃); δ=1.40-1.86 (m, 2H), 2.08 (m, 1H), 2.45 (dd, 1H), 3.29 (m, 1H), 3.82 (s, 3H), 4.25 (ddd, 1H), 4.53 (ddd, 1H), 4.84 (d, 1H), 5.15 (d, 1H), 6.65 (dd, 1H), 6.93 (dd, 1H), 7.21 (d, 1H), 7.64 (t, 1H), 7.87 (d, 1H), 8.18 (s, 1H).

Diastereomer 2

¹H-NMR (300 MHz, CDCl₃); δ=1.40-1.86 (m, 2H), 2.27 (m, 1H), 2.85 (dd, 1H), 2.97 (m, 1H), 3.84 (s, 3H), 4.22 (ddd, 1H), 4.60 (ddd, 1H), 4.65 (d, 1H), 5.43 (d, 1H), 6.78 (dd, 1H), 6.81 (d, 1H), 6.89 (dd, 1H), 7.52 (t, 1H), 7.78 (d, 1H), 8.26 (s, 1H).

### Example 5

### 5-{[5-Hydroxy-5-(trifluoromethyl)-2,3,3a,4,5,6-hexahydrobenzo[de]chromen-6-yl]amino}-quinolin-2(1H)-one

### 5-Aminoquinolin-2(1H)-one

4.5 g 5-nitroquinolin-2(1H)-one (Chem. Pharm. Bull. (1981), 29, pp. 651-56) is hydrogenated with hydrogen in 200 mL ethyl acetate and 500 mL methanol in the presence of 450 mg palladium on activated carbon as the catalyst at standard pressure until achieving a complete reaction. The catalyst is removed by filtration through diatomaceous earth and the reaction solution is concentrated in vacuo, yielding 3.8 g of the title compound as yellow solids.

¹H-NMR (DMSO): δ=5.85 (bs, 2H), 6.27 (d, 1H), 6.33 (d, 1H), 6.43 (d, 1H), 7.10 (t, 1H), 8.07 (d, 1H), 11.39 (br, 1H).

By analogy with Example 1, 600 mg (2.18 mmol) 3-(chroman-4-yl)-2-hydroxy-2-(trifluoromethyl)propanal is reacted with 386 mg (2.39 mmol) 5-aminoquinolin-2(1H)-one to yield the corresponding 5-{[3-(chroman-4-yl)-2-hydroxy-2-(trifluoromethyl) propylidene]amino}quinolin-2(1H)-one. 100 mg imine is placed in 2 mL dichloromethane and treated at 0° C. with 2.4 mL (2.40 mmol) of a 1M TiCl₄ solution. The mixture is allowed to heat up to 0° C. for 60 minutes and the solution is poured onto a mixture of ice and saturated sodium bicarbonate solution and agitated vigorously for 10 minutes. It is extracted with EtOAc, washed with saturated sodium chloride solution and dried over sodium sulfate. By concentrating and performing chromatography on silica gel (dichloromethane/2-propanol 0-5%), 18 and 14 mg desired product are obtained as separated diastereomers.

Diastereomer 1

¹H-NMR (400 MHz, DMSO); δ=1.43 (dd, 1H), 1.60 (dq, 1H), 2.19 (dd, 1H), 2.55 (dd, 1H), 2.98 (m, 1H), 4.02 (t, 1H), 4.28 (ddd, 1H), 4.78 (d, 1H), 6.02 (d, 1H), 6.23 (d+s, 2H), 6.38 (d, 1H), 6.55 (d, 1H), 6.65 (d, 1H), 7.00 (t, 1H), 7.13 (t, 1H), 8.15 (d, 1H), 11.52 (s, 1H).

Diastereomer 2

¹H-NMR (400 MHz, DMSO); δ=1.63 (dq, 1H), 1.80 (dd, 1H), 1.97 (dd, 1H), 2.19 (dd, 1H), 3.15 (m, 1H), 4.15 (td, 1H), 4.38 (ddd, 1H), 5.28 (d, 1H), 6.07 (d, 1H), 6.30 (d, 1H), 6.45 (s, 1H), 6.52 (d, 1H), 6.59 (d, 1H), 6.65 (d, 1H), 6.97 (t, 1H), 7.20 (t, 1H), 8.13 (d, 1H), 11.50 (s, 1H).

The following compounds can be synthesized by a similar procedure:

### Example 6

### 6-[(8-Fluoro-2-methylquinazolin-5-yl)amino]-5-(trifluoromethyl)-2,3,3a,4,5,6-hexahydrobenzo[de]chromen-5-ol

### 5-Amino-8-fluoro-2-methylquinazoline

A solution of 2.4 g (18.6 mmol) 2,5-difluoroaniline in 11 mL water and 1.6 mL concentrated hydrochloric acid (37%) was first stirred for 1 hour at 50° C. and then added to a solution of 3.35 g (20.25 mmol) chloral hydrate and 21.27 g (149.7 mmol) sodium sulfate in 72 mL water. The mixture was agitated for 30 minutes more at RT and then heated to 125° C. over 45 minutes after adding 4.09 g (58.9 mmol) hydroxylammonium chloride in 19 mL water and then kept at this temperature for 5 minutes. After cooling and one more hour, the precipitated light brown solids were filtered out, washed with water and dried, yielding 3.0 g (15.0 mmol) of the hydroxylimine as an intermediate which was dissolved by portions in 15 mL concentrated sulfuric acid at 60° C. After complete addition, the mixture was heated for 2 hours at 80° C. and for 4 hours at 90° C., then allowed to cool. The solution was then poured onto 100 g ice and extracted with ethyl acetate. The organic phase is washed with water, dried over sodium sulfate and concentrated. After chromatography on silica gel with hexane-ethyl acetate (0-45%), 1.2 g (7.1 mmol) 4,7-difluoroisatin is obtained. Over a period of 10 minutes 1.8 mL of a 30% hydrogen peroxide solution is added by drops to isatin in 30 mL of a 1 molar sodium hydroxide solution. After 2 hours of agitation at RT, the mixture is cooled to 0° C. and 5 mL of a 4 molar hydrochloric acid is added and then diluted with 50 mL water. The mixture is extracted with ethyl acetate, dried over sodium sulfate and concentrated, yielding quantitatively 1.27 g of 3,6-difluoroanthranilic acid, which was reacted without further purification. The 3,6-difluoroanthranilic acid is heated to 100° C. for 45 minutes in 8 mL acetic anhydride. After cooling, the resulting acetic acid and excess acetic anhydride is removed as azeotropically with toluene in vacuo. The residue is mixed with 40 mL of a 25% ammonia solution while cooling with ice and agitated for 72 hours, then diluted with water and acidified with acetic acid. The mixture is extracted with ethyl acetate and the organic phase is washed with water, dried over sodium sulfate and concentrated. The resulting 1.03 g (5.25 mmol) 5,8-difluoro-2-methyl-3H-quinazolin-4-one and 6 g phosphorus pentachloride are heated for 12 hours to 125° C. in 20 mL phosphoryl chloride. After cooling, the reaction mixture is poured into saturated NaHCO₃ solution and extracted with ethyl acetate. The organic phase is dried and the solvent is removed, yielding quantitatively 1.7 g 4-chloro-5,8-difluoro-2-methyl-quinazoline, which is dissolved in 60 mL ethyl acetate and 5 mL triethylamine. 600 mg palladium on carbon is added and the mixture is agitated for 2 hours in a hydrogen atmosphere (uptake of 480 mL hydrogen) at standard pressure. The solution is freed of catalyst by filtration through Celite, washing again with 100 mL ethanol and evaporating. After chromatography on silica gel with hexane-ethyl acetate-ethanol (0-40%), 550 mg. 5,8-difluoro-2-methylquinazoline is obtained. To 240 mg (1.3 mmol) 5,8-difluoro-2-methylquinazoline, 300 mg (1.13 mmol) 18-crown-6 in 10 mL DMF is added 890 mg (13.7 mmol) sodium azide and the mixture is heated for 8 hours at 125° C. The solvent is removed in vacuo and the residue is chromatographed on silica gel with ethyl acetate, yielding 52 mg product.

¹H-NMR (300 MHz, CDCl₃); δ=2.92 (s, 3H), 4.31 (br, 2H), 6.67 (dd, 1H), 7.38 (dd, 1H), 9.37 (s, 1H).

### Example 7

### 6-[(7,8-Difluoro-2-methylquinazolin-5-yl)amino]-5-(trifluoromethyl)-2,3,3a,4,5,6-hexahydrobenzo[de]chromen-5-ol

### 5-Amino-7,8-difluoro-2-methylquinazoline

At −70° C., 156 mL (391 mmol) of a 2.5M butyl lithium solution in hexane is added by drops to 41.7 g (180 mmol) 2,2-dimethyl-N-(3,4,5-trifluorophenyl)propionamide in 385 mL THF. The mixture is agitated for 1 hour and then 38.6 mL DMF in 90 mL THF is added by drops, whereupon the solution is allowed to warm up to −60° C. Agitation is continued for 1 more hour at −70° C. and then the cold reaction solution is poured over a mixture of 2 kg ice and 400 mL concentrated hydrochloric acid solution, then agitated vigorously and extracted repeatedly with diethyl ether after 1 hour. The organic phase is washed with water until neutral and dried over sodium sulfate. When concentrated, this yields 49.3 g (188 mmol) crude 4,5,6-trifluoro-2-N-pivaloylaminobenzaldehyde which is added together with 26 g (275 mmol) acetamidine hydrochloride, 38.3 g (277 mmol) potassium carbonate and 30 g molecular sieve (4A) to 206 mL butyronitrile. The mixture is heated for 18 hours at 145° C. while agitating vigorously and the solvent is removed in vacuo. When the residue is chromatographed on silica gel with hexane/ethyl acetate (0-100%), 9:1 g 7,8-difluoro-5-N-pivaloylamino-2-methylquinazoline is obtained.

2.0 g (7.2 mmol) 7,8-difluoro-5-N-pivaloylamino-2-methylquinazoline is dissolved in 140 mL toluene and cooled to −70° C. Over a period of 30 minutes, 24 mL (28.8 mmol) of a 1.2M diisobutylaluminum hydride solution in toluene is added by drops. The reaction mixture is allowed to warm up to −25° C. over 2 hours and then stirred for 2 hours at −25° C. Isopropanol and then water are added slowly and agitated for 12 hours at room temperature until forming a precipitant, which is removed by filtration through Celite. The product is washed well with a methylene chloride-methanol mixture and concentrated. The residue is agitated vigorously in 200 mL ethyl acetate and 50 mL methanol together with 100 g silica gel and 20 g manganese dioxide, then filtered through Celite, washed well with a methylene chloride-methanol mixture and concentrated. When chromatographed on silica gel with hexane-ethyl acetate (0-100%), 370 mg of the product is obtained.

¹H-NMR (300 MHz, CD₃OD); δ=2.81 (s, 3H), 6.64 (dd, 1H), 9.52 (s, 1H).

By analogy with Example 1, 250 mg (0.91 mmol) 3-(chroman-4-yl)-2-hydroxy-2-(trifluoromethyl)propanal is reacted with 200 mg (1.02 mmol) 5-amino-7,8-difluoro-2-methylquinazoline to yield a corresponding 3-(chroman-4-yl)-1-[(7,8-difluoro-2-methylquinazolin-5-yl)imino]-2-(trifluoromethyl)propan-2-ol. 200 mg imine is placed in 4.0 mL dichloromethane and treated with 2.2 mL (2.21 mmol) of a 1M boron tribromide solution at −40° C., then allowed to heat up to 0° C. over a period of 60 minutes. The solution is then poured over a mixture of ice and saturated sodium bicarbonate solution and agitated vigorously for 10 minutes, extracted with EtOAc, washed with saturated sodium chloride solution and dried over sodium sulfate. When concentrated and chromatographed on basic silica gel (hexane/2-propanol 10-20%), it yields 18 mg of the desired product as a mixture of two diastereomers.

Mixture of Diastereomers:

¹H-NMR (300 MHz, CD₃OD, selected signals); δ=0.87 (m, 1H), 1.15-1.30 (m, 3H), 1.42 (s, 3H), 170-2.16 (m, 7H), 3.02-3.13 (m, 1H), 4.00-4.25 (m, 2H), 6.58 (dd, 1H), 6.72 (t, 1H), 6.80 (m, 1H), 6.95-7.18 (m, 2H), 9.47 (s, 1H).

Accessible by a Similar Method:

### Example 8

### 5-{[5-Hydroxy-5-(trifluoromethyl)-2,3,3a,4,5,6-hexahydrobenzo[de]chromen-6-yl]amino}-2-methylphthalazin-1-one

### Example 9

### 6-[(2-Methylquinazolin-5-yl)amino]-5-(trifluoromethyl)-2,3,3a,4,5,6-hexahydrobenzo[de]thiochromen-5-ol

### 2-Hydroxy-3-(thiochroman-4-yl)-2-(trifluoromethyl)propanal

This compound can be synthesized from thiochroman-4-one by analogy with Example 1, using Raney nickel for hydrogenation instead of palladium on carbon as the catalyst.

Mixture of Diastereomers:

¹H-NMR (500 MHz, CD₃OD, selected signals); δ=0.60-0.72 (m, 1H), 1.40-1.65 (m, 2H), 1.75-2.00 (m, 3H), 2.14-2.28 (m, 3H), 2.49 (dd, 1H), 2.87 (m, 1H), 3.00 (m, 1H), 3.04-3.18 (m, 2H), 3.38-3.90 (m, 1H, diastereomer A+B), 6.78 (d, 1H, diastereomer A), 6.85-7.30 (m, 4H, diastereomer A+B), 9.13 (s, 1H, diastereomer A), 9.68 (s, 1H, diastereomer B).

By analogy with Example 1, 183 mg (0.63 mmol) 2-hydroxy-3-(thiochroman-4-yl)-2-(trifluoromethyl)propanal is reacted with 100 mg (0.63 mmol) 5-amino-2-methylquinazoline to yield the corresponding 3-(thiochroman-4-yl)-1-[(2-methylquinazolin-5-yl)imino]-2-(trifluoromethyl)propan-2-ol. 40 mg imine is placed in 2.0 mL dichloromethane and treated at −40° C. with 0.93 mL (0.93 mmol) of a 1M boron tribromide solution, then allowed to heat up to 0° C. over a period of 60 minutes and the solution is poured over a mixture of ice and saturated sodium bicarbonate solution and agitated vigorously for 10 minutes, extracted with EtOAc, washed with saturated sodium chloride solution and dried over sodium sulfate. When concentrated and chromatographed on silica gel (dichloromethane/2-propanol 2-4%), 12 mg of the desired product is obtained as a mixture of two diastereomers.

Mixture of Diastereomer:

¹H-NMR (300 MHz, CD₃OD, selected signals); δ=1.25-1.34 (m, 2H), 1.56-75 [sic; 1.75] (m, 1H), 1.95 (t, 1H), 2:22-2.34 (m, 1H), 2.38 (dd, 1H), 2.78 (s, 3H), 2.93-3.10 (m, 2H), 3.22 (dd, 1H), 4.59 (s, 1H, diastereomer A), 5.40 (s, 1H, diastereomer B), 6.95-7.09 (m, 4H), 7.68 (t, 1H, diastereomer B), 7.78 (t, 1H, diastereomer A), 9.50-9.17 (m, 1H).

Accessible by a Similar Method:

### Example 10

### 2-methyl-6-[(2-methylquinazolin-5-yl)amino]-5-(trifluoromethyl)-2,3,3a,4,5,6-hexahydrobenz[de]thiochromen-5-ol

### 2-Hydroxy-3-(2-methylthiochroman-4-yl)-2-(trifluoromethyl)propanal

This can be synthesized by analogy with Example 1 from 6-chloro-2-methylthiochroman-4-one using Raney nickel in hydrogenation instead of palladium on carbon as the catalyst.

### Example 11

### 5-[(5-Hydroxy-5-(trifluoromethyl)-2,3,3a,4,5,6-hexahydrobenzo[de]chromen-6-yl]amino}-phthalazin-1 (2H)-one

By analogy with Example 1, 600 mg (2.18 mmol) 3-(chroman-4-yl)-2-hydroxy-2-(trifluoromethyl)propanal is reacted with 384 mg (2.39 mmol) 5-amino-2H-phthalazin-1-one to yield the corresponding 5-[3-(chroman-4-yl)-2-hydroxy-2-(trifluoromethyl)propylidene]amino}phthalazin-[(2H)-one. 210 mg imine is placed in 5 mL dichloromethane and treated with 2.5 mL (2.5 mmol) of a 1M BBr₃ solution at 40° C. The mixture is allowed to heat up to 0° C. over a period of 60 minutes and the solution is then poured over a mixture of ice and saturated sodium bicarbonate solution, stirred for 10 minutes, extracted with EtOAc, washed with saturated sodium chloride solution and dried over sodium sulfate. When concentrated and chromatographed on RP silica gel (MeCN/water 38-50%), this yields 69 and 34 mg of the desired product as separate diastereomers.

Diastereomer 1:

¹H-NMR (300 MHz, DMOS-d₆); δ=1.49 (dd, 1H) 1.63 (qd, 1H), 2.18 (dd, 1H), 2.54 (dd, 1H), 2.98 (m, 1H), 4.03 (t, 1H), 4.29 (ddd, 1H), 4.88 (d, 1H), 6.20 (s, 1H), 6.41 (d, 1H), 6.68 (dd, 1H), 6.98 (q, 1H), 7.40 (d, 1H), 7.47 (t, 1H), 8.58 (s, 1H), 12.48 (s, 1H).

Diastereomer 2:

¹H-NMR (300 MHz, DMSO-d₆); δ=1.63 (qd, 1H), 1.81 (t, 1H), 1.99 (m, 1H), 2.21 (dd, 1H), 3.10-3.20 (m, 1H), 4.16 (td, 1H), 4.48 (ddd, 1H), 5.37 (d, 1H), 6.43 (s, 1H), 6.48 (d, 1H), 6.60 (d, 1H), 6.18 (d, 1H), 6.98 (t, 1H), 7.24 (d, 1H), 7.39 (d, 1H), 7.53 (t, 1H), 8.55 (s, 1H), 12.48 (s, 1H).

### Example 12

### 5-{[9-Fluoro-5-hydroxy-3a-methyl-5-(trifluoromethyl)-2,3,3a 4,5,6-hexahydrobenzo[de]chromen-6-yl]amino}quinolin-2(1H)-one

### 2-Hydroxy-3-(4-methylchroman-4-yl)-2-trifluoromethylpropionaldehyde

7.3 g E/Z-(chroman-4-ylidene)ethyl acetate (J. Med. Chem. 2001, 44, pp. 1085-1098), 100 mg copper(I) chloride and 5.1 mL chlorotrimethylsilane in 55 mL THF are mixed slowly at 0° C. with 11.5 mL methyl magnesium chloride solution (3.3M in THF) so the temperature always remains below 5° C. The mixture is stirred for 1 hour more at 0° C. and 10 hours at room temperature. The batch is mixed with saturated ammonium chloride solution and extracted with ether. The combined organic phases are washed with brine, dried with sodium sulfate and concentrated in vacuo. After chromatography on silica gel (hexane/ethyl acetate 100:0→90:10), this yields 2-(4-methylchroman-4-yl) ethyl acetate as a raw product, which is then mixed with 1 g lithium aluminum hydride in 50 mL THF at 0° C. and agitated for 1.5 hours at 0° C. The batch is cautiously poured onto saturated ammonium chloride solution and diluted with ethyl acetate, then filtered through diatomaceous earth: The aqueous phase is extracted with ethyl acetate. The combined organic phases are washed with brine, dried with sodium sulfate and concentrated in vacuo. When chromatographed on silica gel (hexane/ethyl acetate 100:0→60:40) this yields 1.2 g 2-(4-methylchroman-4-yl)ethanol as a color oil. 0.58 mL oxalyl chloride in 25.0, mL dichloromethane is mixed at −78° C. with 1.0 mL DMSO in 25.0 mL dichloromethane. After 5 minutes, 1.2 g 2-(4-methylchroman-4-yl)ethanol is added by drops to 25.0 mL dichloromethane at −78° C. After 15 minutes, this is mixed with 4 mL triethylamine and heated slowly to room temperature, then washed with water, brine, 1% sulfuric acid and saturated sodium bicarbonate solution, dried with sodium sulfate and evaporated in vacuo. After performing chromatography on silica gel (hexane/ethyl acetate 100:0→90:10), this yields 970 mg 2-(4-methylchroman-4-yl)ethanal as a colorless oil. A solution of 870 mg 2-(4-methylchroman-4-yl)ethanal and 2 mL trifluoromethyl trimethylsilane in 30 mL THF is mixed with 0.87 mL tetrabutylammonium fluoride solution (1M in THF) at 0° C. and agitated for 1 hour. Then a spatula tip of solid tetrabutylammonium fluoride is also added and mixed with water. Extraction is performed with ethyl acetate and the organic phase is washed with brine and dried with sodium sulfate, yielding 1.2 g 2-(4-methylchroman-4-yl)-1-trifluoromethylethanol as a brown oil (mixture of diastereomers). 7.8 g Dess-Martin periodinane in 100 mL dichloromethane is mixed with a solution of 1.2 g 2-(chroman-4′-yl)-1-trifluoromethylethanol in 20 mL dichloromethane at 0° C. and agitated for 1 hour at 0° C. The batch is mixed with saturated sodium bicarbonate solution, diluted with diethyl ether and agitated with sodium thiosulfate solution for 30 minutes. The organic phase is separated and washed with water, then dried with sodium sulfate and evaporated in vacuo, yielding 1.1 g 3-(4-methylchroman-4-yl)-1,1,1-trifluoropropan-2-one as a brown oil. 9.2 mL vinyl magnesium bromide solution (1H in THF) is mixed t room temperature with a solution of 1.1 g 3-(4-methylchroman-4-yl)-1,1,1-trifluoropropane-2-one in 30 mL THF. The mixture is agitated for 5 hours at room temperature and then poured onto saturated ammonium chloride solution.

The batch is extracted with ethyl acetate and the combined organic phases are washed with brine, dried with sodium sulfate and concentrated in vacuo. When chromatographed on silica gel (hexane/ethyl acetate 100:0→95:5), this yields 930 g 1-(4-methylchroman-4-yl)-2-trifluoromethyl-3-buten-2-ol as an oil. Working at −70° C., ozone is introduced into a solution of 900 mg 1-(4-methylchroman-4-yl)-2-trifluoromethyl-3-buten-2-ol in 56 mL dichloromethane and 14 mL methanol at −70° C. until the solution assumes a blue color. Then argon is passed through the solution for 1 minute, the solution is then mixed with 0.42 mL dimethyl sulfide and agitated for 1 hour more at −70° C., then heated slowly to room temperature and agitated for 10 hours more. The batch is poured over water and extracted with dichloromethane. The combined organic phases are dried and concentrated in vacuo. When chromatographed on silica gel (hexane/ethyl acetate 100:0→96:4), this yields 330 mg 2-hydroxy-3-(4-methylchroman-4-yl)-2-trifluoromethylpropionaldehyde as a yellow oil (mixture of diastereomers).

¹H-NMR (CDCl₃, selected signals); δ=1.38 (s, 3H, diastereomer A), 1.43 (s, 3H, diastereomer B), 1.57-1.65 (m, 2H), 1.81 (ddd, 1H), 2.13 (ddd, 1H), 2.37 (d, 1H, diastereomer B), 2.45-2.60 (m), 2.83 (d, 1H, diastereomer B), 3.69 (s, 1H, diastereomer A), 3.86 (s, 1H, diastereomer B), 3.97 (dd, 1H, diastereomer B), 4.08-4.35 (m), 6.78-6.84 (m, 1H, diastereomer A+B), 6.88-6.95 (m, 1H), 7.07-7.21 (m), 7.23-7.30 (m), 8.88 (s, 1H, diastereomer B), 9.56 (s, 1H, diastereomer A).

By analogy with Example 1, 300 mg (1.0 mmol) 2-hydroxy-3-(4-methylchroman-4-yl)-2-(trifluoromethyl)propionaldehyde is reacted with 160 mg (1.0 mmol) 5-aminoquinolin-2(1H)-one to yield a corresponding 5-{[2-hydroxy-3-(4-methylchroman-4-yl)-2-(trifluoromethyl)propylidene]amino}quinolin-2(1H)-one. 250 mg imine is placed in 3 mL dichloromethane and treated with 3.0 mL (3.0 mmol) of a 1M BBr₃ solution at −40° C. The mixture is allowed to heat up to 0° C. over a period of 60 minutes and the solution is poured onto a mixture of ice and saturated sodium bicarbonate solution and stirred vigorously for 10 minutes, then extracting with EtOAc, washed with saturated sodium chloride solution and dried over sodium sulfate. When concentrated and chromatographed on silica gel (dichloromethane/1-PrOH 0-6%), this yields 81 and 16 mg, respectively, of the desired product as separated diastereomers.

Diastereomer 1:

¹H-NMR (600 MHz, DMSO-d₆); δ=1.40 (s, 3H), 1.84 (m, 2H), 1.93 (d, 1H), 2.38 (d, 1H), 4.28 (m, 2H), 5.04 (d, 1H), 6.13 (s, 1H), 6.15 (d, 2H), 6.24 (d, 1H), 6.43 (d, 1H), 6.60 (d, 1H), 6.72 (d, 2H), 7.04 (t, 1H), 7.12 (t, 1H), 8.20 (d, 1H), 11.60 (s, 1H).

Diastereomer 2:

¹H-NMR (600 MHz, DMSO-d₆); δ=1.51 (s, 3H), 1.76 (m, 1H), 1.88 (dd, 1H), 2.00 (d, 1H), 2.18 (d, 1H), 4.33 (m, 1H), 4.41 (m, 1H), 5.26 (d, 1H), 6.18 (d, 1H), 6.34 (s, 1H), 6.37 (d, 1H), 6.48 (d, 1H), 6.57 (d, 1H), 6.68 (d, 1H), 6.70 (d, 1H), 7.01 (t, 1H), 7.22 (t, 1H), 8.24 (d, 1H), 11.67 (s, 1H).

### Example 13

### 5-{[9-Fluoro-5-hydroxy-5-(trifluoromethyl)-2,3,3a,4,5,6-hexahydrorobenzo[de]thiochromen-6-yl]amino}quinolin-2(1H)-one

By analogy with Example 1, 700 mg (2.4 mmol) 2-hydroxy-3-(thiochroman-4-yl)-2-(trifluoromethyl)propanal is reacted with 386 mg (2.4 mmol) 5-aminoquinolin-2(1H)-one to yield a corresponding 5-{[2-hydroxy-3-(thiochroman-4-yl)-2-(trifluoromethyl)propylidene]amino}quinolin-2(1H)-one. 100 mg imine is placed in 3 mL dichloromethane and treated with 2.3 mL (2.3 mmol) of a 1M BBr₃ solution at −40° C., then allowed to warm up to 0° C. over a period of 60 minutes. The solution is then poured over a mixture of ice and saturated sodium bicarbonate solution and agitated vigorously for 10 minutes, then extracted with EtOAc, washed with saturated sodium chloride solution and dried over sodium sulfate. After concentrating and chromatographing on silica gel (dichloromethane/i-PrOH 2-4%), this yields 28 mg of the desired product as a mixture of diastereomers.

Mixture of Diastereomers:

¹H-NMR (500 MHz, DMSO-d₆, selected signals); δ=1.57 (ddd, 1H, diastereomer A+B), 1.96 (t, 1H), 2.25 (m, 1H, diastereomer A+B), 3.88-3.99 (m, 1H, diastereomer A+B), 3.07 (ddd, 1H), 3.13-3.27 (m, 1H, diastereomer A+B), 4.32 (d, 1H, diastereomer A), 4.57 (br. s, 1H, diastereomer B), 5.47 (d, 1H, diastereomer B), 6.10 (d, 1H, diastereomer A), 6.37 (d, 1H, diastereomer A), 6.40 (s, diastereomer A), 6.58 (d, 2H, diastereomer A+B), 6.90 (d, 1H, diastereomer A), 7.05 (m, 2H, diastereomer A+B), 7.25 (t, 1H), 8.16 (d, 1H), 11.53 (s, 1H, diastereomer A+B).

### Example 14

### 6-[(8-Fluoro-2-methylquinazolin-5-yl)amino]-5-(trifluoromethyl)-2,3,3a,4,5,6-hexahydrobenzo[de]thiochromen-5-ol

By analogy with Example 1, 328 mg (1.1 mmol) 2-hydroxy-3-(thiochroman-4-yl)-2-(trifluoromethyl)propanal is reacted with 200 mg (1.1 mmol) 5-amino-8-fluoroquinazoline to form the corresponding 1-[(8-fluoro-2-methylquinazolin-5-yl)imino]-3-(thiochroman-4-yl)-2-(trifluoromethyl)propan-2-ol. 185 mg imine is placed in 5 mL dichloromethane and treated with 4.1 mL (4.1 mmol) of a 1M BBr₃ solution at −40° C. The mixture is allowed to heat up to 0° C. over a period of 60 minutes and the solution is poured over a mixture of ice and saturated sodium bicarbonate solution and agitated vigorously for 10 minutes, then extracted with EtOAc, washed with saturated sodium chloride solution and dried over sodium sulfate. When concentrated and chromatographed on silica gel (dichloromethane/i-PrOH 2-4%) and performing chromatography again on basic silica gel, this yields 18 mg of the desired product as a mixture of diastereomers.

Mixture of Diastereomers:

¹H-NMR (400 MHz, CD₃OD, selected signals): δ=1.12 (t, 1H), 1.15-1.38 (m, 2H, diastereomer A+B), 1.53-1.68 (m, 1H), 1.90 (t, 1H), 2.25 (m, 1H), 2.36 (dd, 1H), 2.73-2.85 (m, 4H), 2.86-3.08 (m, 2H), 3.10-3.33 (m, 2H), 5.31 (s, 1H), 6.88 (dd, 1H), 6.90-7.05 (m, 4H), 7.46 (dd, 1H), 7.52 (dd, 1H), 9.54-9.68 (m, 1H, diastereomer A+B).

### Example 15

### 6-[(7,8-Difluoro-2-methylquinazolin-5-yl)amino]-5-(trifluoromethyl)-2,3,3a,4,5,6-hexahydrobenzo[de]thiochromen-5-ol

By analogy with Example 1′, 223 mg (0.77 mmol) 2-hydroxy-3-(thiochroman-4-yl)-2-(trifluoromethyl)propanal is reacted with 150 mg (0.77 mmol) 5-amino-7,8-difluoroquinazoline to yield the corresponding 1-[(7,8-difluoro-2-methylquinazolin-5-yl)imino]-3-(thiochroman-4-yl)-2-(trifluoromethyl)propan-2-ol. 90 mg imine is placed in 4 mL dichloromethane and treated with 1.9 mL (1.9 mmol) of a 1M BBr₃ solution at −40° C. The mixture is allowed to heat up to 0° C. over a period of 60 minutes and the solution is poured over a mixture of ice and saturated sodium bicarbonate solution and then agitated vigorously for 10 minutes. Extraction is performed with EtOAc, then washed with saturated sodium chloride solution and dried over sodium sulfate. After concentrating and performing chromatography on silica gel (dichloromethane/1-PrOH 2-4%), this yields 13 mg of the desired product.

¹H-NMR (300 MHz, MeOD): δ=1.63 (qd, 1H), 1.94 (dd,1H), 2.25 (m, 1H), 2.33 (dd, 1H), 2.78 (s, 3H), 2.90-3.10 (m, 2H), 3.23 (td, 1H), 5.34 (s, 1H), 6.85 (dd, 1H), 7.00 (s, 3H), 9.54 (s, 1H).

### Example 16

### 6-[(7-Fluoro-2-methylquinazolin-5-yl)amino]-5-(trifluoromethyl)-2,3,3a,4,5,6-hexahydrobenzo[de]thiochromen-5-ol

By analogy with Example 1, 246 mg (0.85 mmol) 2-hydroxy-3-(thiochroman-4-yl)-2-(trifluoromethyl)propanal is reacted with 150 mg (0.85 mmol) 5-amino-7-fluoroquinazoline to yield the corresponding 1-[(7-fluoro-2-methylquinazolin-5-yl)imino]-3-(thiochroman-4-yl)-2-(trifluoromethyl)propan-2-ol. 70 mg imine is placed in 4 mL dichloromethane and treated with 1.56 mL (1.56 mmol) of a 1M BBr₃ solution at −40° C. The mixture is allowed to heat up to 0° C. over a period of 60 minutes and the solution is poured over a mixture of ice and saturated sodium bicarbonate solution and then agitated vigorously for 10 minutes, then extracted with EtOAc, washed with saturated sodium chloride solution and dried over sodium sulfate. After concentrating and performing chromatography on silica gel (dichloromethane/1-PrOH 2-4%), this yields 13 mg of the desired product in the form of a mixture of two diastereomers.

Mixture of Diastereomers:

¹H-NMR (300 MHz, MeOD, selected signals); δ8=1.20-1.36 (m, 1H), 1.55-1.75 (m, 1H), 1.95 (t, 1H), 2.24 (m, 1H), 2.37 (dd, 1H), 2.74 (m, 3H), 2.88-3.10 (m, 2H), 3.20 (dd, 1H), 5.48 (s, 1H), 6.68-6.80 (m, 2H), 6.90-7.04 (m, 2H), 7.20-7.45 (m, 1H), 9.40-9.55 (m, 1H).

The following compounds can be synthesized by similar methods, using the amino described above, in WO 2005/034939 or in WO 2003/082827:
5-{[9-Fluoro-5-hydroxy-3a-methyl-5-(trifluoromethyl)-2,3,3a,4,5,6-hexahydrobenzo[de]chromen-6-yl]amino}isoquinolin-1(2H)-one
5-{[9-Fluoro-5-hydroxy-3a-methyl-5-(trifluoromethyl)-2,3,3a,4,5,6-hexahydrobenzo[de]chromen-6-yl]amino}-2-methylphthalazin-1-one
6-[(7,8-Difluoro-2-methylquinazolin-5-yl)amino-2-methyl-5-(trifluoromethyl)-2,3,3a,4,5,6-hexahydrobenzo[de]thiochromen-5-ol
6-[(Indazol-4-yl)amino]-2-methyl-5-(trifluoromethyl)-2,3,3a,4,5,6-hexahydrobenzo[de]thiochromen-5-ol
6-[(8-Fluoro-2-methylquinazolin-5-yl)amino]-3a-methyl-5-(trifluoromethyl)-2,3,3a,4,5,6-hexahydrobenzo[de]thiochromen-5-ol
6-[(8-Fluoro-2-methylquinazolin-5-yl)amino]-2-methyl-5-(trifluoromethyl)-2,3,3a,4,5,6-hexahydrobenzo[de]thiochromen-5-ol
9-Fluoro-6-[(isoquinolin-5-yl)amino]-5-(trifluoromethyl)-2,3,3a,4,5,6-hexahydrobenzo[de]chromen-5-ol
4-{(9-Fluoro-5-hydroxy-5-(trifluoromethyl)-2,3,3a,4,5,6-hexahydrobenzo[de]chromen-6-yl]amino}-1,3-dihydroindol-2-one
4-{(5-Hydroxy-5-(trifluoromethyl)-2,3,3a,4,5,6-hexahydrobenzo[de]thiochromen-6-yl]-amino}-1,3-dihydroindol-2-one
9-Fluoro-6-[(1-methylindazol-4-yl)amino]-5-(trifluoromethyl)-2,3,3a,4,5,6-hexahydrobenzo[de]chromen-5-ol
5-{[9-Fluoro-5-hydroxy-5-(trifluoromethyl)-2,3,3a,4,5,6-hexahydrobenzo[de]chromen-6-yl]amino}-2-methylisoquinolin-1-one
5-{[5-Hydroxy-2-methyl-5-(trifluoromethyl)-2,3,3a,4,5,6-hexahydrobenzo[de]thiochromen-6-yl]amino}-1-methylquinolin-2-one
5-{[9-Fluoro-5-hydroxy-5-(trifluoromethyl)-2,3,3a,4,5,6-hexahydrobenzo[de]chromen-6-yl]amino}-2-methylphthalazin-1-one
5-{[9-Fluoro-5-hydroxy-5-(trifluoromethyl)-2,3,3a,4,5,6-hexahydrobenzo[de]chromen-6-yl]amino}isoquinolin-1 (2H)-one
5-{[5-Hydroxy-3a-methyl-5-(trifluoromethyl)-2,3,3a,4,5,6-hexahydrobenzo[de]thiochromen-6-yl]amino}-2-methylphthalazin-1-one
6-[(2-Methylquinazolin-5-yl)amino]-3a-methyl-5-(trifluoromethyl)-2,3,3a,4,6,6-hexahydrobenzo[de]thiochromen-5-ol

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102004063227.8, filed Dec. 22, 2004 and U.S. Provisional Application Ser. No. 60/642,519, filed Jan. 11, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Stereoisomers of general formula (I) 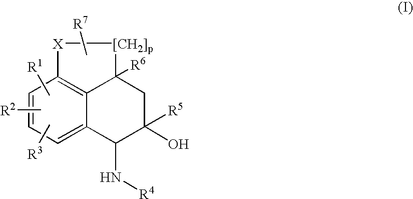
wherein
R¹ and R² independently of one another denote a hydrogen atom, a hydroxyl group, a halogen atom, an optionally substituted (C₁-C₁₀) alkyl group, a (C₁-C₁₀) alkoxy group, a (C₁-C₁₀) alkylthio group, a (C₁-C₅) perfluoroalkyl group, a cyano group, a nitro group or
R¹ and R² together may form a group selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁—NH—N═CH—, where n=1 or 2 and the terminal atoms are linked to directly vicinal ring carbons
or NR⁵R⁹, where R⁸ and R⁹ independently of one another may denote hydrogen, C₁-C₅ alkyl or (CO)—C₁-C₅ alkyl,
R³ denotes a hydrogen atom, a hydroxyl group, a halogen atom, an optionally substituted (C₁-C₁₀) alkyl group, an (C₁-C₁₀) alkoxy group, a (C₁-C₁₀) alkylthio group, a (C₁-C₅) perfluoroalkyl group, a cyano group,
R⁴ denotes a C₁-C₁₀ alkyl group or a substituted C₁-C₁₀ alkyl group with substituents of one or more groups selected from 1 to 3 hydroxyl groups, halogen atoms or 1 to 3 (C₁-C₅) alkoxy groups; an optionally substituted (C₃-C₇) cycloalkyl group, an optionally substituted heterocyclyl group,
an optionally substituted aryl group; a mono- or bicyclic heteroaryl groups optionally containing 1 to 4 nitrogen atoms and/or 1 to 2 oxygen atoms and/or 1 to 2 sulfur atoms and/or 1 to 2 keto groups, optionally substituted by one or more groups selected from (C₁-C₅) alkyl groups (which may optionally be substituted by 1 to 3 hydroxyl or 1 to 3 COOR¹⁰ groups where R¹⁰ denotes C₁-C₆ alkyl or benzyl), (C₁-C₅) alkoxy groups, hydroxyl groups, halogen atoms, (C₁-C₃) exoalkylidene groups, where this group may be linked to the amine of the tricyclic system at any position and may optionally be hydrogenated at one or more positions,
R⁵ denotes a (C₁-C₅) alkyl group or an optionally partially or completely fluorinated (C₁-C₅) alkyl group, a (C₃-C₇) cycloalkyl group, a (C₃-C₇) cycloalkyl(C₁-C₈)alkyl group, a (C₃-C₇) cycloalkyl(C₂-C₈)alkenyl group, a heterocyclyl group, a heterocyclyl(C₁-C₈)alkyl group, heterocyclyl(C₂-C₈)alkenyl group, an aryl group, an aryl(C₁-C₈)alkyl group, (an aryl(C₂-C₈)alkenyl group, an aryl(C₂-C₈)alkynyl group; a mono- or bicyclic heteroaryl group containing one or more nitrogen atoms and/or oxygen atoms and/or sulfur atoms and optionally substituted by one or more keto groups, (C₁-C₅) alkyl groups, (C₁-C₅) alkoxy groups, halogen atoms, (C₁-C₃) exoalkylidene groups;
a heteroaryl (C₁-C₈) alkyl group or
a heteroaryl (C₂-C₈) alkenyl group,
where these groups may be linked to the chromene system at any position and may optionally be hydrogenated in one or more positions,
R⁶ and R⁷ independently of one another denote a hydrogen atom, a halogen atom, a (C₁-C₅) alkyl group which may be substituted with OR⁸, SR⁸, NR⁸R⁹,
p is 1, 2 or 3 and
X is an oxygen atom, a sulfur atom, a CH₂ group or an NR⁹ group.

2. Stereoisomers of general formula (I) according to claim 1 wherein
R¹ and R² independently of one another denote a hydrogen atom, a hydroxyl group, a halogen atom, an optionally substituted (C₁-C₁₀)alkyl group, a (C₁-C₁₀) alkoxy group, a (C₁-C₁₀) alkylthio group, a (C₁-C₅) perfluoroalkyl group, a cyano group, a nitro group or
R¹ and R² together may form a group selected from the groups —O—(CH₂)ₙ—O—, —O—(CH₂)ₙ—CH₂—, —O—CH═CH—, —(CH₂)ₙ₊₂—, where n=1 or 2 and the terminal atoms are linked to directly vicinal ring carbons,
or NR⁸R⁹, where R⁸ and R⁹ independently of one another denote hydrogen, C₁-C₅ alkyl or (CO)—C₁-C₅ alkyl,
R³ denotes a hydrogen atom, a hydroxyl group, a halogen atom, an optionally substituted (C₁-C₁₀) alkyl group, an (C₁-C₁₀) alkoxy group, a (C₁-C₁₀) alkylthio group, a (C₁-C₅) perfluoroalkyl group, a cyano group,
R⁴ denotes a C₁-C₁₀ alkyl group or a substituted C₁-C₁₀ alkyl group with substituents of one or more groups selected from 1 to 3 hydroxyl groups, halogen atoms, 1 to 3 (C₁-C₅) alkoxy groups; an optionally substituted phenyl group; a mono- or bicyclic heteroaryl group containing 1 to 3 nitrogen atoms and/or 1 to 2 oxygen atoms and/or 1 to 2 sulfur atoms and/or 1 to 2 keto groups, optionally substituted by 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups, where these groups may be linked to the amine of the tricyclic system at any position and may optionally be hydrogenated at one or more positions,
R⁵ denotes a (C₁-C₅) alkyl group or an optionally partially or completely fluorinated (C₁-C₅) alkyl group, an aryl group, an aryl(C₁-C₈)alkyl group, an aryl(C₂-C₈)alkenyl group, a (C₃-C₇)cycloalkyl group, a (C₃-C₇)cycloalkyl(C₁-C₈)alkyl group, a (C₃-C₇)cycloalkyl(C₂-C₈) alkenyl group
R⁶ and R⁷ independently of one another denote a hydrogen atom, a halogen atom, a methyl or ethyl group which may be substituted with OR⁸, SR⁸, N(R⁹)₂,
p is 1, 2 or 3 and
X is an oxygen atom, a sulfur atom, a CH₂ or an NR⁹ group.

3. Stereoisomers of general formula (I) according to claim 1 wherein
R¹ and R² independently of one another denote a hydrogen atom, a hydroxyl group, a halogen atom, an optionally substituted (C₁-C₅) alkyl group, a (C₁-C₅) alkoxy group, a (C₁-C₅) perfluoroalkyl group, a cyano group, a nitro group,
R³ denotes a hydrogen atom, a hydroxyl group, a halogen atom, an optionally substituted (C₁-C₁₀) alkyl group or a (C₁-C₁₀) alkoxy group,
R⁴ denotes a (C₁-C₁₀) alkyl group, a (C₁-C₁₀) alkyl group substituted by 1 to 3 hydroxyl groups or halogen atoms; a phenyl, naphthyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group, optionally substituted by one or more groups selected from 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups,
where these groups may be linked to the amine of the ring system at any position and may optionally be hydrogenated at one or more positions,
R⁵ denotes a (C₁-C₅) alkyl group or an optionally partially or completely fluorinated (C₁-C₅) alkyl group,
R⁶ and R⁷ independently of one another denote a hydrogen atom, a halogen atom, a methyl or ethyl group which may be substituted with OR⁸, SR⁸, N(R⁹)₂, where R⁸ and R⁹ independently of one another may denote hydrogen, C₁-C₅ alkyl or (CO)—C₁-C₅ alkyl,
p is 1, 2 or 3
X is an oxygen atom, a sulfur atom, a CH₂ or an NR⁹ group.

4. Stereoisomers of general formula (I) according to claim 1, wherein
R¹, R² and R³ independently of one another denote a hydrogen atom, a hydroxyl group, a halogen atom, an optionally substituted (C₁-C₁₀) alkyl group, a (C₁-C₁₀) alkoxy group, a cyano group,
R⁴ denotes a mono- or bicyclic heteroaryl group containing 1 to 3 nitrogen atoms and/or 1 to 2 oxygen atoms and/or 1 to 2 sulfur atoms and/or 1 to 2 keto groups and optionally substituted by one or more groups selected from 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups,
where this group may be linked to the amine of the ring system at any position and may optionally be hydrogenated at one or more positions,
R⁵ denotes a (C₁-C₅) alkyl group or an optionally partially or completely fluorinated (C₁-C₅) alkyl group,
R⁷ and R⁸ [sic; R⁶ and R⁷] independently of one another denote a hydrogen atom, a halogen atom, a methyl or ethyl group,
p is 1 or 2
X is an oxygen or sulfur atom.

5. Stereoisomers of general formula (I) according to claim 1, wherein
R¹, R² and R³ independently of one another denote a hydrogen atom, a hydroxyl group, a halogen atom, an optionally substituted (C₁-C₁₀) alkyl group, a (C₁-C₁₀) alkoxy group, a cyano group,
R⁴ denotes a phenyl, naphthyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group,
optionally substituted by one or more groups selected from 1 to 2 keto groups, 1 to 2 (C₁-C₅) alkyl groups, 1 to 2 (C₁-C₅) alkoxy groups, 1 to 3 hydroxyl groups, 1 to 3 halogen atoms or 1 to 2 (C₁-C₃) exoalkylidene groups,
where these groups may be linked to the amine of the ring system at any position and may optionally be hydrogenated at one or more positions,
R⁵ denotes a (C₁-C₅) alkyl group or an optionally partially or completely fluorinated (C₁-C₅) alkyl group,
R⁷ and R³ [sic; R⁶ and R⁷] independently of one another denote a hydrogen atom, a halogen atom, a methyl or ethyl group,
p is ¹ or 2
X is an oxygen atom or a sulfur atom.

6. Use of the stereoisomers according to claim 1 to produce a pharmaceutical drug.

7. Use of the stereoisomers of claim 1 to produce a pharmaceutical drug for treatment of inflammatory diseases.

8. Pharmaceutical preparations containing at least one stereoisomer according to claim 1 or mixtures thereof and pharmaceutically tolerable vehicles.

9. Stereoisomers of general formula (I) according to claim 1 in the form of salts with physiologically tolerable anions.

10. Method for synthesis of the stereoisomers of general formula (I) wherein the radicals have the meanings defined in claim 1, unless otherwise indicated, characterized in that either
a) stereoisomers of general formula (III) 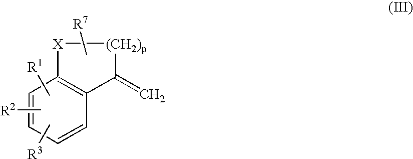
wherein R¹, R², R³, R⁷, X and p have the meanings given in claim 1, are converted to compounds of general formula (IV) 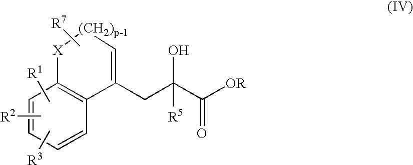
by an optionally enantioselective En reaction with α-keto acids R⁵(CO)COOR¹⁰ in the presence of optionally chiral Lewis acids,
the radical R⁶═H is introduced by hydrogenation and by reduction and reaction with amines of formula R⁴—NH₂,
where R⁴ has the meanings given in claim 1,
the compounds of formula (V) are synthesized 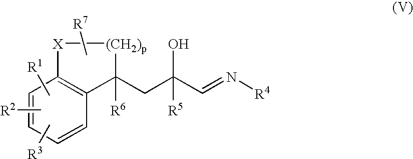
wherein R¹, R², R³, R⁴, R⁵, R⁷, X and p have the meanings given in claim 1, which are then cyclized to yield compounds of general formula (I) either without another reagent or by adding organic or inorganic acids or Lewis acids at temperatures of −70° C. to 80° C. or
b) compounds of general formula (VI) synthesized by methods with which those skilled in the art are familiar 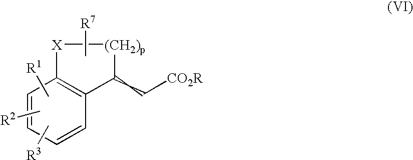
are converted to compounds of general formula (VII) by reduction of a double bond or by reaction with a cuprate reagent containing R⁶, where R⁶ has the meanings given in claim 1, reduction of the esters and optional oxidation, 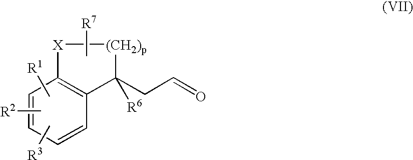
which is then reacted with a silicone compound of general formula C_{q}F_{2q+1}—Si(CH₃)₃, where q=1, 2, 3 or 4, in the presence of a catalyst or with a metal organyl of the formula R⁵A, where R⁵ has the meanings given in claim 1 and A is magnesium halogen or lithium, to yield a compound of general formula (VIII) 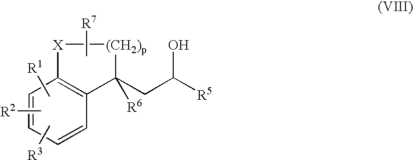
which is converted to a compound of general formula (X) by oxidation and reaction with a compound of the formula M-CN, where M may stand for sodium, potassium, copper or trimethylsilyl 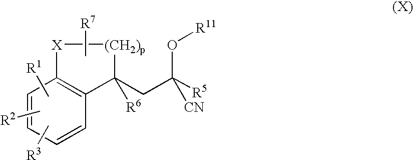
which is then converted to a compound of general formula (XI) after reduction 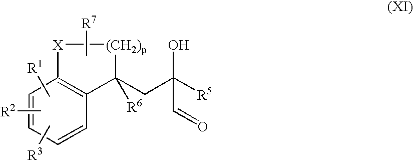
which can be reacted and cyclized with an amine R⁴—NH₂ by analogy with method a) or
c) compounds of general formula (VIII) 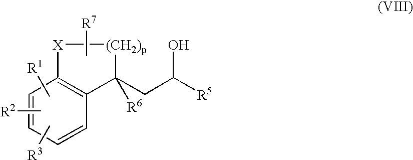
are reacted with alkenyl metal organyl R¹²R¹³(C)↑CH-A where A denotes magnesium halogen or lithium and R¹², R¹³ denote hydrogen or C₁-C₆ alkyl, to generate compounds of general formula (XII) 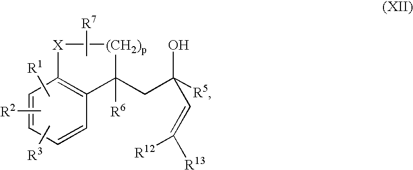
which can be converted to compounds of general formula (X) by oxidative cleavage of the double bond, and compounds can then be reacted by analogy with variant b) to form compounds of general formula (I).

11. Method step for synthesis of stereoisomers of general formula (I) according to claim 10 characterized in that stereoisomers of general formula
(V) 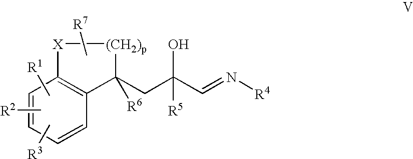
wherein R¹, R², R³, R⁴, R⁵, R⁶, R⁷, X and p have the meanings defined above, are cyclized with the addition of organic or inorganic acids or Lewis acids.

12. Compounds of general formula (V) according to claim 11 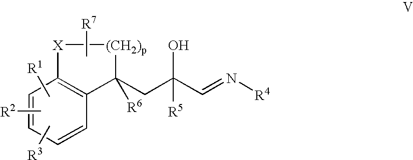
wherein R¹, R², R³, R⁴, R⁵, R⁶, R⁷, X and p have the meanings defined above.

13. Method step for synthesis of compounds of general formula (I) according to claim 10, characterized in that stereoisomers of general formula (IV) 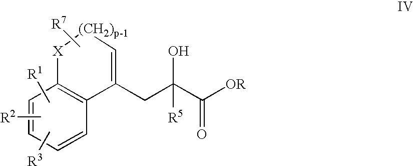
are reduced and reacted with a corresponding amine of formula R⁴—NH₂ to yield the imine of general formula (V).

14. Compounds of formula (IV) according to claim 13, wherein R¹, R², R³, R⁵, X and p have the meanings defined above.

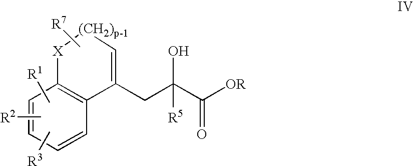

15. Compounds of formula (III) according to claim 10 wherein R¹, R², R³, R⁷, X and p have the meanings defined above

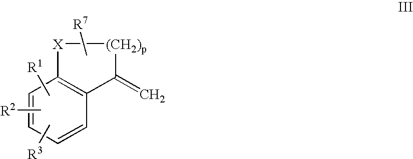

16. Amines according to claim 10, 5-amino-7-fluoro-2-methylquinazoline, 5-amino8-fluoro-2-methylquinazoline and 5-amino-7,8-difluoro-2-methylquinazoline.
